(19) United States
(12) Patent Application Publication
(10) Pub. No.: US 2020/0330644 A1
(43) Pub. Date: Oct. 22, 2020

(54) METHODS OF FORMING THREE-DIMENSIONAL TISSUES SCAFFOLDS USING BIOLOGICAL FIBER INKS AND METHODS OF USE THEREOF

(71) Applicants: PRESIDENT AND FELLOWS OF HARVARD COLLEGE, Cambridge, MA (US); SOGANG UNIVERSITY, Seoul (KR)

(72) Inventors: Luke Alexander MacQueen, Cambridge, MA (US); Kevin Kit Parker, Cambridge, MA (US); Kwanwoo Shin, Seoul (KR); Giho Choi, Yongsan-gu (KR)

(21) Appl. No.: 16/756,214

(22) PCT Filed: Oct. 16, 2018
(86) PCT No.: PCT/US2018/056074

§ 371 Date: Apr. 15, 2020

Related U.S. Application Data
(60) Provisional application No. 62/572,695, filed on Oct. 16, 2017.

(51) Int. Cl. A61L 27/18; A61L 27/20; A61L 27/22; A61L 27/56; C12M 1/12

(52) U.S. Cl. CPC A61L 27/18; A61L 27/20; B33Y 10/00; A61L 27/56; C12M 25/14; A61L 27/222

(57) ABSTRACT

Some embodiments provide method of forming a three-dimensional tissue scaffold that includes extruding a bioink material through a nozzle onto a support while moving the nozzle relative to the support or moving the support relative to the nozzle to form a three-dimensional structure of the bioink material. The bioink material includes a plurality of polymeric fibers, each polymeric fiber having a diameter on a range of 0.1 μm to 20 μm, and each polymeric fiber comprising one or more biocompatible polymers, and a carrier. The method also includes cross-linking or heat fusing at least some of plurality of polymeric fibers in the three-dimensional structure of the bioink material.

### RELATED APPLICATIONS

This application claims the benefit of priority to U.S. Provisional Application No. 62/572,695, filed on Oct. 16, 2017, the entire contents of which are incorporated herein by reference.

### BACKGROUND OF THE INVENTION

The current need for organ and tissue replacement, tissue repair and regeneration for patients is continually growing such that supply is not meeting the high demand primarily due to a paucity of donors as well as biocompatibility issues that lead to immune rejection of the transplant. In an effort to overcome these drawbacks, scientists working in the field of tissue engineering and regenerative medicine have investigated the use of scaffolds as an alternative to transplantation. In addition to use for tissue replacement, tissue repair and regeneration, engineered tissues that recapitulate biological tissues are also useful for in vitro testing of pharmacological agents and for in vitro disease models. However, significant limitations associated with the conventional tissue engineering biomaterials used to prepare such scaffolds include unpredictable bioprotein content (e.g., decellularized tissues), insufficient structural resolution (e.g., bioprinting), and difficulty balancing cell infiltration with scaffold degradation (e.g., hydrogels). For example, current tissue or organ printing methods, limited by print resolution, cannot print microscale and nanoscale features found in natural extra cellular matrix (ECM), limiting the fidelity with which printed tissue scaffolds recapitulate natural tissues.

Accordingly, there is a need in the art for methods of preparing suitable tissue engineering scaffolds.

### SUMMARY OF THE INVENTION

The present invention is based, at least in part, on the discovery of methods to form stable and resilient two- and three-dimensional scaffolds using a bioink material extruded through a nozzle onto a support that can, e.g., recapitulate microscale and nanoscale features found in natural extracellular matrix (ECM), that can, e.g., modulate immune responses and enhance tissue regeneration, and are thus useful for, e.g., regenerative therapies.

Accordingly, in one aspect, the present invention provides a method of forming a three-dimensional tissue scaffold. The methods include extruding a bioink material through a nozzle onto a support while moving the nozzle relative to the support or moving the support relative to the nozzle to form a three-dimensional structure of the bioink material, the bioink material comprising: a plurality of polymeric fibers, each polymeric fiber having a diameter on a range of 0.1 μm to 20 μm, and each polymeric fiber comprising one or more biocompatible polymers; and a carrier; and cross-linking or heat fusing at least some of plurality of polymeric fibers in the three-dimensional structure of the bioink material.

Each of the plurality of polymeric fibers may have a length of less than 3 mm.

An average length of a polymeric fiber in the plurality of polymeric fibers may be in a range of 0.05 mm and 0.3 mm; in a range of 0.07 mm to 0.25 mm; and/or in a range of 0.9 mm and 2.2 mm. In some embodiments, the plurality of polymeric fibers includes polymeric fibers having lengths less than 0.05 mm.

The nozzle may have an inner diameter in a range of 0.01 mm to 0.6 mm.

In some embodiments, a length of each of the plurality of polymeric fibers may be less than 0.25 mm, an average length of a polymeric fiber in the plurality of polymeric fibers may be between 0.05 mm and 0.25 mm, and the plurality of polymeric fibers includes polymeric fibers having lengths less than 0.05 mm.

In some embodiments, an average length of a polymeric fiber in the plurality of polymeric fibers is in a range of 0.15 mm to 0.25 mm.

In other embodiments, an average length of a polymeric fiber in the plurality of polymeric fibers is in a range of 0.18 to 0.22 mm.

In yet other embodiments, an average length of a polymeric fiber in the plurality of polymeric fibers is in a range of 0.05 mm and 1.5 mm.

An average length of a polymeric fiber in the plurality of polymeric fibers may be in a range of 0.8 mm to 1.2 mm.

A ratio of a weight of the polymeric fibers to a weight of the carrier in the bioink material may be less than 20.

An inner diameter of the nozzle may be in a range of 0.4 mm to 2.5 mm, wherein a maximum length of a polymeric fiber in the plurality of polymeric fibers is greater than 0.25 mm and smaller than the inner diameter of the nozzle, wherein the plurality of polymeric fibers includes polymeric fibers having lengths less than 0.05 mm, and wherein a ratio of a weight of the polymeric fibers to a weight of the carrier in the bioink material is less than one.

In some embodiments, each of the plurality of polymeric fibers has a length less than 0.05 mm.

In some embodiments, at least some of the plurality of the polymeric fibers anisotropically align along a direction of extrusion in the three-dimensional structure of the fiber material.

In some embodiments, two or more of an inner diameter of the nozzle, an average length of polymeric fiber in the plurality of polymeric fibers, a maximum length of a polymeric fiber in the plurality of polymeric fibers, a ratio of weight of polymeric fibers to weight of carrier in the bioink material and a composition of the plurality of polymeric fibers are selected such that at least some of the plurality of polymeric fibers anisotropically align along a direction of extrusion in the three-dimensional structure of the fiber material.

The bioink material is extruded at ambient temperature.

The methods of the invention may, in some embodiments, further comprise providing the bioink material.

In some embodiments, providing the bioink material includes disposing the plurality of polymeric fibers in the carrier less than about 30 minutes prior to a beginning of extruding the bioink material onto the support.

In other embodiments, providing the bioink material includes: mechanically breaking down a polymeric fibrous material to reduce average polymeric fiber length;

fractionating a slurry including the polymeric fibrous material to obtain a desired distribution of polymeric fiber lengths; drying the fractionated slurry leaving the plurality of polymeric fibers having the desired distribution of polymeric fiber lengths; and suspending the plurality of polymeric fibers having the desired distribution of polymeric fiber lengths in the carrier.

Cross-linking may be chemical cross-linking or enzymatic cross-linking.

In some embodiments, exposure to UV radiation is used before, during, or after cross-linking or heat fusing for increased resistance to degradation.

In some embodiments, the methods of the invention further comprise removing some or all of the carrier after cross-linking or heat fusing at least some of the plurality of polymeric fibers.

The resulting three-dimensional scaffold prepared according to the methods of the invention may include a tube-shaped structure.

In some embodiments, at least some of the plurality of polymeric fibers are anisotropically aligned in a circumferential direction.

In other embodiments, the resulting three-dimensional scaffold prepared according to the methods of the invention may define a cavity.

In some embodiments, at least some of the plurality of polymeric fibers are aligned with a circumference of the cavity.

The carrier may comprise a hydrogel forming solution.

In some embodiment, the extrusion is into a bath on the support wherein the bath includes an agent that interacts with the hydrogel forming solution to form a hydrogel.

In some embodiments, the carrier comprises one or more of alginate, or gelatin.

In some embodiments, the bioink material further comprises additional agents for cell programming. Such additional agents for cell programming may be disposed on or in the plurality of polymeric fibers.

In other embodiments, the bioink material further comprises one or more biologically active agents. The one or more biologically active agents may be disposed on or in the plurality of polymeric fibers.

In yet other embodiments, the bioink material further comprises one or more pharmaceutically active agents. The one or more pharmaceutically active agents may be disposed on or in the plurality of polymeric fibers.

In some embodiments, the bioink material further comprises cells.

In other embodiments, the bioink material further comprises fluorescent molecules. The fluorescent molecules may be disposed on or in the plurality of polymeric fibers.

In some embodiments, the bioink material further comprises nanoparticles.

The movement of the nozzle relative to the support or movement of the support relative to the nozzle to form a three-dimensional structure of the bioink material may include using a 3-D printing system or additive manufacturing system to control relative movement of the nozzle and the support.

In one aspect, the present invention provides a method of forming a three-dimensional engineered tissue. The methods include providing a three-dimensional tissue scaffold produced according to the methods of the invention; seeding the scaffold with cells; and culturing the cells under suitable conditions to form a tissue, thereby forming a three-dimensional engineered tissue.

In another aspect, the present invention provides a method of forming an engineered food product. The methods include providing a three-dimensional tissue scaffold produced according to the methods of the invention; seeding the scaffold with muscle cells; and culturing the cells under suitable conditions to form a muscle tissue, thereby forming a three-dimensional engineered food product.

In one aspect, the present invention provides a bioink material for use with a three-dimensional printer or an additive manufacturing system. The bioink includes a plurality of polymeric fibers having an average length in a range of 0.07 mm to 0.25 mm, each polymeric fiber having a diameter on a range of 0.05 mm and 0.3 mm, and each polymeric fiber comprising one or more biocompatible polymers; and a carrier comprising a hydrogel forming solution, wherein the average length of the plurality of polymeric fibers results in at least some of the polymeric fibers being preferentially oriented along an extrusion direction when the bioink is extruded from a three-dimensional printer or additive manufacturing system.

Each of the plurality of polymeric fibers may have a length of less than 3 mm.

The plurality of polymeric fibers may have an average length in a range of 0.07 mm to 0.25 mm; and/or an average length in a range of 0.9 mm and 2.2 mm.

In some embodiments, the plurality of polymeric fibers includes polymeric fibers has lengths less than 0.05 mm.

The bioink may be extrudable at ambient temperature.

In some embodiments, the biolink material may be extrudable at temperatures less than 50° C. In some embodiments, the biolink material may be extrudable at a temperature in a range of 40° C. to 50° C.

In some embodiments, the bioink material may be extrudable at a temperature in a range of 5° C. to 20° C.

In some embodiments, the carrier comprises one or more of alginate or gelatin.

In some embodiments, the one or more biocompatible polymers include one or more of gelatin, hyaluronic acid, and polycaprolactone.

In one aspect, the present invention provides a kit for forming the bioink material of the invention. The kit includes the plurality of polymeric fibers; and a carrier forming material such that mixing the carrier forming material with water forms the carrier.

### BRIEF DESCRIPTIONS OF THE DRAWINGS

The features and advantages of the present disclosure will be more fully understood from the following description of exemplary embodiments when read together with the accompanying drawings. The drawings are intended to illustrate the teachings taught herein and are not intended to show relative sizes and dimensions unless otherwise noted, or to limit the scope of examples or embodiments. In the drawings, the same numbers are used throughout the drawings to reference like features and components of like function.

FIG. 1 is a flow chart schematically depicting a method for forming a tissue scaffold (e.g., a three-dimensional tissue scaffold) in accordance with some embodiments.

FIG. 2 schematically depicts a bioink material being extruded from a nozzle onto a support and fibers aligned with an extrusion direction in accordance with some embodiments.

FIG. 3A schematically depicts a cantilever structure that can be extruded with fibers aligned longitudinally with the extrusion direction in accordance with some embodiments.

FIG. 3B schematically depicts a cantilever structure that can be extruded with a first extrusion layer including fiber primarily aligned with a width of the cantilever and a second extrusion layer including fibers aligned longitudinally in accordance with some embodiments.

FIG. 3C schematically depicts an ellipsoid structure that can be extruded with fibers in an outer layer and in an inner layer aligned circumferentially in accordance with some embodiments.

FIG. 3D schematically depicts an ellipsoid structure that can be extruded with fibers in an outer layer oriented circumferentially and fibers in an inner layer aligned longitudinally in accordance with some embodiments.

FIG. 3E schematically depicts a tube structure that can be extruded with fibers in an outer layer oriented circumferentially and fibers in an inner layer aligned longitudinally in accordance with some embodiments.

FIG. 4 is a flow chart schematically depicting a method of forming a bioink material in accordance with some embodiments.

FIG. 5A includes images of a gelatin fiber material and the material after freeze drying and cutting in accordance with an example embodiment.

FIG. 5B includes images of the freeze dried and cut polymeric fiber material being ground in solvent to form a gelatin fiber slurry for filtering or fractionation to obtain a desired distribution of lengths in accordance with an example embodiment.

FIG. 6A includes microscope images of gelatin fibers having a desired length distribution dispersed in solvent at different concentrations in accordance with an example embodiment.

FIG. 6B includes (i) a microscope image of a bioink material extruded filament (with bioprinter insert) and a higher resolution microscope image of the bioink material extruded and gelled filament showing gelatin fibers (indicated with arrows) within the bioink extruded filament aligned with the extrusion axis at a relatively low fiber concentration and ii) a microscope image of a bioink material including a relatively high concentration of gelatin fibers extruded from a needle without gelling of the carrier and a microscope image after cross-linking in accordance with two example embodiments.

FIG. 7 includes a microscope image of an extruded bioink material including gelatin fibers in an alginate carrier without gelling of the carrier where the gelatin fibers have diameters on the order of about 10 μm and a length of about 1 mm in accordance with an example embodiment.

FIG. 8A includes a microscope image of an extruded line of bioink material including an ungelled alginate carrier loaded with a high density of gelatin fibers having a long average fiber length of greater than 2 mm showing fiber alignment with the axis of extrusion in accordance with an example embodiment.

FIG. 8B includes a microscope image of the extruded line after being dried in a solution containing salts where the salt crystallization sites are determined by gelatin fiber features, giving rise to regularly spaced salt dendrites sprouting from the gelatin fibers, demonstrating that that extrusion conditions determine fiber alignment, and subsequent chemical reactions guided by fibers in accordance with an example embodiment.

FIG. 9 includes images of AlgHA filaments formed by extruding a bioink including hyaluronic acid HA fibers in an alginate solution carrier into baths having different concentrations of CaCl₂ for gelation of the carrier in accordance with example embodiments.

FIG. 10 includes a microscope image of filaments produced by extrusion of a bioink material including HA fibers in an alginate solution carrier into a CaCl₂ bath for gelation of the carrier and a detail showing an individual HA fiber in the resulting filament in accordance with an example embodiment.

FIG. 11A includes an image of a 3D printed mesh structure from extrusion of the HA and gelatin bioink into a CaCl₂ bath and a detail of the 3D printed mesh structure in the bath in accordance with an example embodiment.

FIG. 11B includes images of the 3D printed mesh structure of FIG. 11A removed from the bath including images at varying magnifications in accordance with an example embodiment.

FIG. 11C is a microscope image of the same 3D printed mesh of FIG. 11B with arrows indicating an extrusion direction and showing fibers preferentially aligning with the print direction in accordance with an example embodiment.

FIG. 12 includes images at various magnifications of sheets formed by printing a HA fiber and gelatin bioink material having a relatively high fiber concentration into a CaCl₂ bath showing peeling of the sheets from the substrate and peeling along the print direction accordance with an example embodiment.

FIG. 13A includes image of freestanding tubes printed using an HA fiber and alginate carrier bioink including (i) tubes printed in a dish, (ii) tubes printed on slides, and (iii) a magnified view of a single printed tube in accordance with example embodiments.

FIG. 13B includes images at various magnifications of a cross-section of a printed tube of FIG. 13A showing circumferential HA fiber alignment along the print direction in accordance with an example embodiment.

FIG. 14A is an image of a 3D printed scale model of a heart ventricle formed using the HA fiber and alginate bioink in accordance with an example embodiment.

FIG. 14B is a microscope image of a cross-section of the scale model of the heart ventrical taken near the apex showing HA fiber alignment near the apex.

FIG. 14C includes a microscope image of a cross-section of the scale model of the heart ventricle taken at about half the height of the ventricle showing circumferential fiber alignment and a detail of that image with edge detection applied to highlight the HA fibers.

FIG. 15 is a table of results of evaluation of the effects of different fiber length distributions and fiber concentrations on printability and fiber anisotropy in resulting structures for gelatin fiber bioinks in accordance with some example embodiments.

FIG. 16 is a table of results of evaluation of the effects of different fiber length distributions and fiber concentrations on printability and fiber anisotropy in resulting structures for HA fiber bioinks in accordance with some example embodiments.

FIG. 17 is a table of results of evaluation of the effects of different fiber length distributions and fiber concentrations on printability and fiber anisotropy in resulting structures for PCL fiber bioinks in accordance with some example embodiments.

### DETAILED DESCRIPTION OF THE INVENTION

Some methods described herein employ biological fiber ink materials (hereafter “bioink materials”) to form a tissue scaffold (e.g., a three-dimensional tissue scaffold). The bioink materials include dispersed biocompatible polymeric material fibers in a carrier that can be extruded (e.g., 3D printed) to form a two-dimensional or three-dimensional structure of the bioink material. The polymeric fibers each have a diameter in a nanometer to micron range (e.g., 0.1 μm to 20 μm). The polymeric fibers are crosslinked (e.g., chemically, enzymatically, etc.) in the extruded structure to form a two-dimensional or three-dimensional tissue scaffold. In some embodiments, at least some of the polymeric fibers in the bioink material preferentially anisotropically align with the extrusion direction (also described herein as the printing direction) enabling control of an orientation of the polymeric fibers and the corresponding microscale and nanoscale features in the resulting two-dimensional or three-dimensional tissue scaffold, e.g., to recapitulate extracellular matrix features found in vivo.

In some embodiments, the bioink materials flow at room temperature and are suitable for use in a wide variety of established and emerging additive manufacturing and bio-printing platforms.

Some methods described herein are methods for forming tissue engineering and regenerative medicine scaffolds that contain anisotropic cross-linked biocompatible micro- or nano-dimension polymeric fibers and that direct cell phenotypes, and their assembly into functional tissues. In some embodiments, the bioinks described herein include biocompatible micro-or nano-scale biocompatible synthetic or natural polymeric fibers dispersed in a carrier that can be extruded to form a three-dimensional structure with controlled anisotropy of the polymeric fibers where the polymeric fibers can be cross-lined to form a stable three-dimensional tissue scaffold structure with micro-scale or nano-scale features that recapitulate biological structures. Crosslinking the fibers within the extruded (e.g., 3D printed) structure after extrusion enables stable and resilent structures to be formed with the control and flexibility provided by current 3D printing and additive manufacturing technologies. In some embodiments, methods are used to form nanofibrous tissue scaffolds that modulate immune responses and enhance tissue regeneration, which may fulfill unmet needs in regenerative therapies.

In some embodiments, the bioink material also includes various additional agents (e.g., biologically active molecules such as peptides, proteins, lipids, nucleotides;

pharmaceutically active agents; fluorescent molecules), living cells (e.g., stem cells, muscle cells), nanoparticles, or any combination of the aforementioned. In some embodiments, any of the aforementioned can be disposed in or on the polymeric fibers. In some embodiments, living cells may be disposed in the carrier material.

In some embodiments, by including additional agents (e.g., cell instructive factors) within the polymeric fibers, and forming a three-dimensional structure of cross-linked polymeric fibers with controlled anisotropy, the resulting two-dimensional or three-dimensional tissue scaffolds with controlled-release of cell-instructive factors may promote beneficial immune system reactions and endogenous repair mechanisms within regenerative medicine applications. They may promote cell reprogramming and tissue genesis within broader tissue engineering applications (e.g., in vitro disease models).

In some embodiments, at least some or all of the carrier is removed from the two-dimensional or three-dimensional structure of cross-linked polymeric fibers (e.g., by incubation in ionic solvents) prior to use of the tissue scaffold.

In some embodiments, storage of the two-dimensional or three-dimensional structure of cross-linked polymeric fibers in the carrier material may extend a shelf life of the tissue scaffold.

In some embodiments, the carrier is or includes a hydrogel forming solution (e.g., a solution for forming a polysaccharide hydrogel such as an alginate or gelatin hydrogel) and the biocompatible polymeric fibers include a polysaccharide (e.g., hyaluronic acid, gelatin), a bioprotein (e.g., collagen type 1 fibrils), or a biocompatible synthetic polymer (e.g, polycaprolactone (PCL)).

### I. Definitions

In order that the present invention may be more readily understood, certain terms are first defined. In addition, it should be noted that whenever a value or range of values of a parameter are recited, it is intended that values and ranges intermediate to the recited values are also part of this invention.

In the following description, for purposes of explanation, specific numbers, materials and configurations are set forth in order to provide a thorough understanding of the invention. It will be apparent, however, to one having ordinary skill in the art that the invention may be practiced without these specific details. In some instances, well-known features may be omitted or simplified so as not to obscure the present invention. Furthermore, reference in the specification to phrases such as “one embodiment” or “an embodiment” means that a particular feature, structure or characteristic described in connection with the embodiment is included in at least one embodiment of the invention. The appearances of phrases such as “in one embodiment” in various places in the specification are not necessarily all referring to the same embodiment.

The articles “a” and “an” are used herein to refer to one or to more than one (i.e., to at least one) of the grammatical object of the article. By way of example, “an element” means one element or more than one element.

The term “comprising” or “comprises” is used herein in reference to compositions, methods, and respective component(s) thereof, that are essential to the disclosure, yet open to the inclusion of unspecified elements, whether essential or not.

The term “consisting of” refers to compositions, methods, and respective components thereof as described herein, which are exclusive of any element not recited in that description of the embodiment.

The word “nozzle” as used herein refers to any element having an opening or orifice for extrusion of a material. The term “nozzle” as used herein, includes, but is not limited to 3D printing nozzles, additive manufacturing nozzles, hollow needles suitable for extrusion, hollow cylinders suitable for extrusion, components of 3-D printing and additive manufacturing heads suitable for extrusion, an orifice in a wall suitable for extrusion.

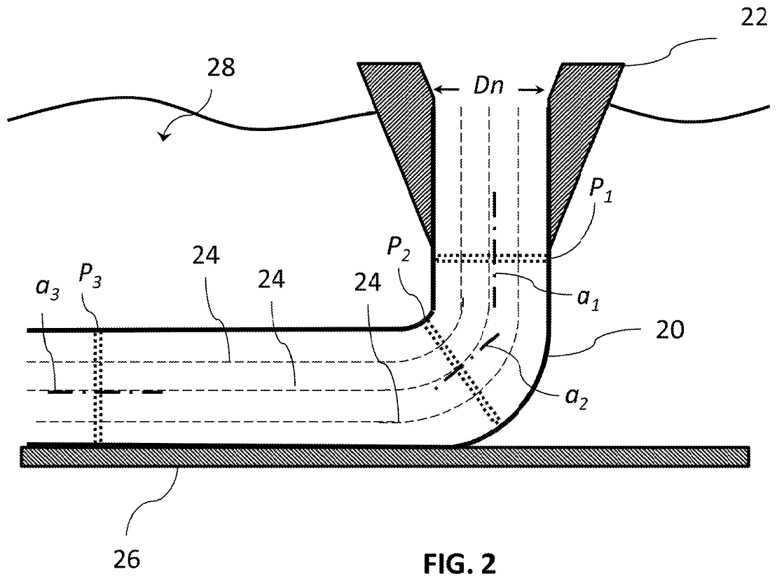

The terms “extrusion axis” or “extrusion direction” refer to a particular axis for each portion (e.g., cross-section) of material that was extruded through the nozzle at a particular point in time. At the time point of extrusion for a particular portion of material, the extrusion axis for that particular portion of material is generally an axis that extends through and is perpendicular to a plane of the nozzle opening. After extrusion, as that particular portion of material moves away from the nozzle and is tilted and/or rotated, the corresponding extrusion axis is tilted or rotated accordingly. This concept is graphically illustrated in FIG. 2 for portions P₁, P₂ and P₃ and respective corresponding extrusion axes a₁, a₂ and a₃. Because interaction with the substrate and movement of the nozzle relative to substrate usually causes these rotations and tilts of each portion of the extruded material and the corresponding rotations and tilts of the extrusion axis for the portion, the extrusion axis for a portion of material may also be referred to as a printing direction for the portion of material.

### II. Methods of the Invention

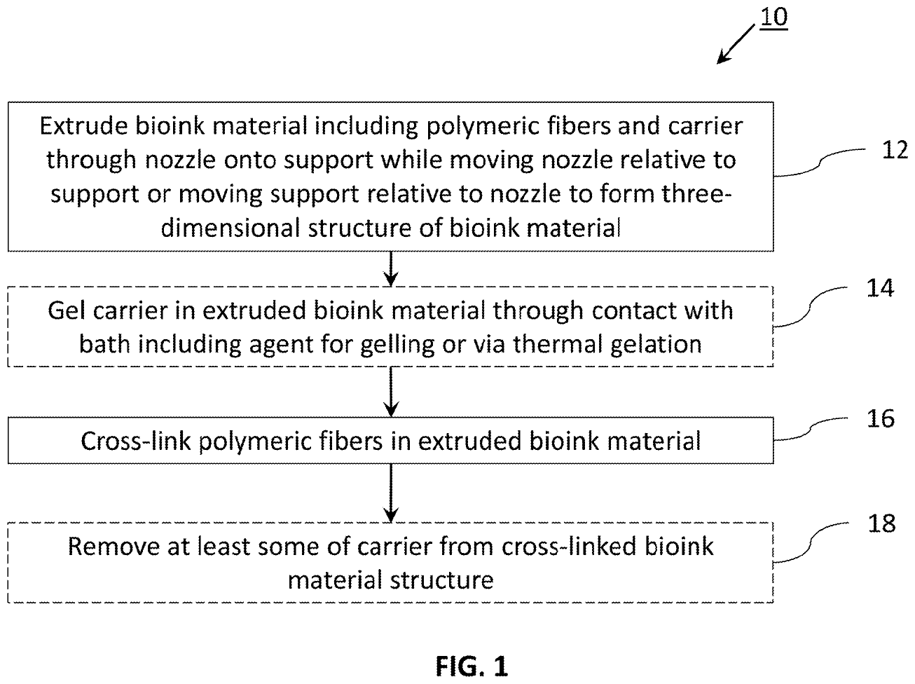

FIG. 1 is a flow chart of a method 10 of forming a tissue scaffold, for example, a three-dimensional tissue scaffold. Although some methods are described herein with respect to forming a three-dimensional structure and a corresponding three-dimensional tissue scaffold, one of ordinary skill it in art in view of the present disclosure would understand that the same methods can be employed to form a two-dimensional structure having a substantially linear or substantially planar shape. The method 10 includes extruding a bioink material through a nozzle onto a support while moving the nozzle relative to the support or moving the support relative to the nozzle to form a three-dimensional structure of the bioink material (12). In some embodiments, movement of the nozzle relative to the support or movement of the support relative to the nozzle to form a three-dimensional structure of the bioink material includes using a 3-D printing system or additive manufacturing system to control relative movement of the nozzle and the support. In some embodiments, the nozzle is part of the 3-D printing system or additive manufacturing system. In some embodiments, the nozzle is a nozzle of a printing head. In some embodiments, the bioink material is extruded at ambient temperature. In some embodiments, the bioink material does not need to be heated for extrusion.

The bioink material includes a plurality of polymeric fibers and a carrier. Each polymeric fiber has a diameter between about 0.1 μm to about 20 μm and comprises one or more biocompatible polymers. Examples of biocompatible polymers for the polymeric fibers are provided below. In some embodiments, the carrier includes a hydrogel forming solution. Examples of materials for the carrier are provided below.

In some embodiments, the bioink material is extruded into a bath including an agent that interacts with the hydrogel forming solution to gel the carrier, or the bioink is extruded into a bath or onto a support having a different temperature than the bioink material for thermal gelation (14). In some embodiments, the bath includes one or more agents that interact with the hydrogel forming solution to gel the carrier. In some embodiments, different agents in the batch cause gelling at different time scales. Examples of agents that interact with the hydrogel forming solution to gel the carrier are provided below. In some embodiments, the bath has a different temperature than the carrier material and the gelation is due, at least in part, to a temperature change in the carrier material after extrusion into the bath. In some embodiments, the gelation is ionic gelation. In some embodiments, the gelation is thermal gelation. In some embodiments, the gelation includes ionic and thermal gelation. The box for this step 14 is indicated with dashed lines as not all embodiments need include this step here. For example, in some embodiments the carrier may include a hydrogel or be gelled prior to extrusion. Although step 14 is shown occurring after step 12 in the flow chart, where the bioink material is extruded into a bath including an agent, or at least one agent, for gelling, the gelling may begin to occur before the extrusion of the three-dimensional structure ends, meaning that there would be temporal overlap between steps 12 and 14.

At least some of the polymeric fibers are cross-linked in the extruded three-dimensional structure (16). In some embodiments, the polymeric fibers are cross-linked after the three-dimensional structure is extruded. Although step 16 is shown occurring after step 12 in the flow chart, in some embodiments, the polymeric fibers are cross-linked as the three-dimensional structure is being extruded, e.g., by extrusion into a cross-linking solution, which means that there is temporal overlap between step 12 and step 16. The cross-linking may be chemical, may be enzymatic, may be via radiation, and/or via other mechanisms and methods. Examples of cross-linking methods and mechanisms for various types of polymeric fibers are provided below.

In some embodiments, some, or all, of the carrier is removed from the three-dimensional extruded structure after cross-linking (18). This step is shown in dashed lines to indicate that it may not be included in some embodiments. In some embodiments, at least some of the carrier forms cross-links in the cross-linked structure and is not removed after cross-linking. In some embodiments, the carrier is left in the structure of crosslinked fibers for storage, and may be subsequently removed in whole or in part prior to use. In some embodiments, the carrier is not removed from the structure after crosslinking. For example, in some embodiments the gelled carrier may contain cells (i.e., a mix of hydrogel and cells, where the hydrogel can be a range of things like gelatin, alginate, etc.). In this case, cells may align and anchor to the embedded fibers and gradually replace their ‘carrier’ with cell-secreted, e.g., extracellular matrix, during culture to produce, e.g., a tissue.

In accordance with some embodiments, the combination of fibers and carrier for the bioink material are selected based on their differential gelation and/or crosslinking conditions such that the mixture of the fibers and carrier is printable and such that the embedded fibers can be thermally bonded or crosslinked independently from the carrier. In some embodiments, where the carrier is at least partially removed from the structure after cross-linking, a reversible carrier sol-gel transition can be achieved independently from fibers embedded in the carrier.

In some embodiments, the polymeric fibers are anisotropically oriented in the extruded bioink material. Specifically, at least some of polymeric fibers are preferentially oriented along an extrusion direction in the extruded three-dimensional structure. This enables control of the polymeric fiber orientation in the resulting three-dimensional structure through the extrusion or printing pattern used to form the three-dimensional structure. FIG. 2 schematically depicts bioink material 20 being extruded from a nozzle 22 having an inner diameter Dn onto a support 26 with dashed lines 24 schematically representing polymeric fibers aligned with the extrusion direction. In some embodiments the bioink material is extruded into a bath 28 including an agent for gelling the carrier. Portions of the extruded material P₁, P₂, P₃ extruded at three different times are indicated with dotted boxes and a corresponding extrusion axis a₁, a₂, a₃ for each respective portion is indicated with dotted and dashed lines in FIG. 2.

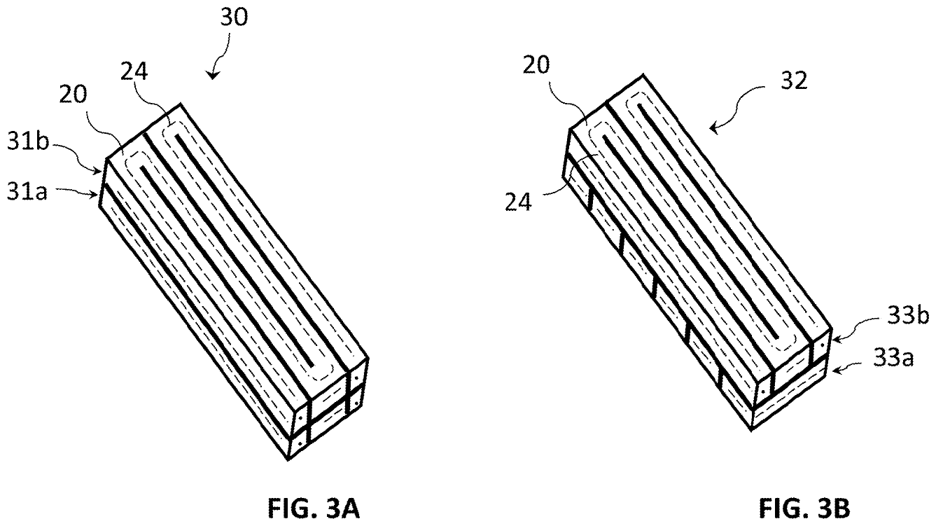

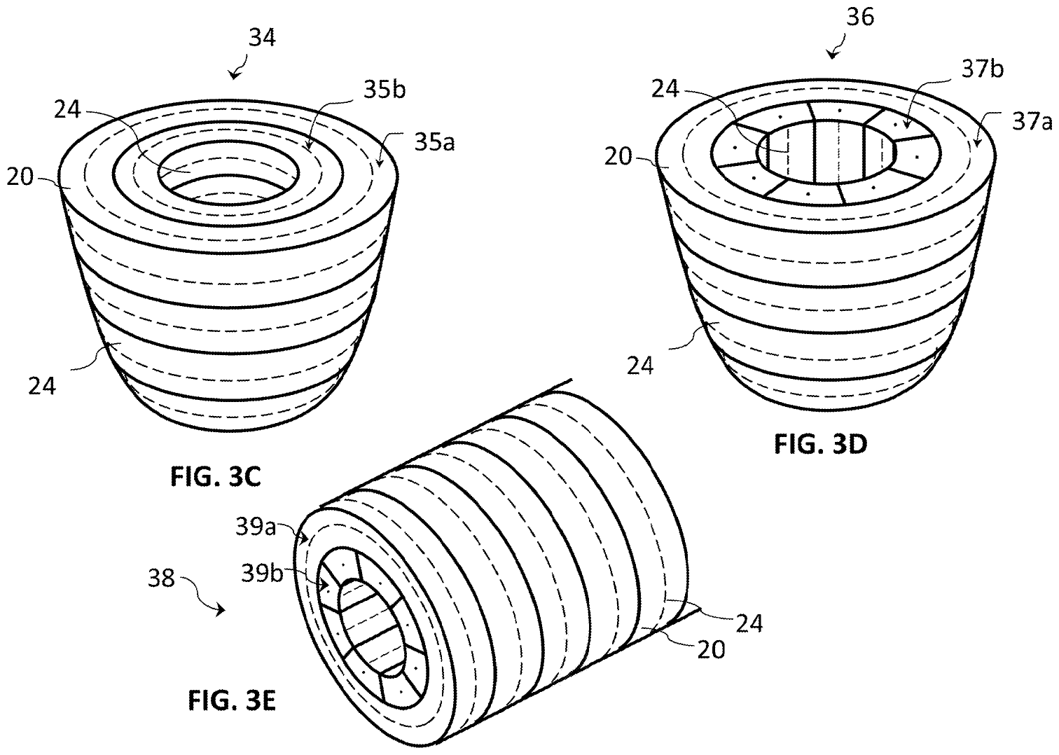

FIGS. 3A-3E schematically depict some example structures that can be extruded using the bioink material 20 and schematically depicts preferential alignment of some fibers 24 along the extrusion direction and a resulting alignment pattern. Structure 30 is in FIG. 3A a cantilever structure with preferential alignment of some fibers along a longitudinal axis of the cantilever. As depicted, both a first extrusion layer 31a and a second extrusion layer 31b are extruded in a pattern in which a direction of extrusion mostly aligns with the longitudinal axis of the cantilever, which results in preferential alignment of fibers along the longitudinal axis for both the first extrusion layer 31a and the second extrusion layer 31b. FIG. 3B depicts a cantilever structure 32 in which the bottom, first extrusion layer 33a is extruded such that the extrusion direction is primarily aligned with a width of the cantilever preferentially orienting the fibers in the bottom, first, extrusion layer along a width of the cantilever 32. The second, top, extrusion layer 33b is extruded such that the extrusion direction and corresponding fiber alignment is preferentially along a length of the cantilever. Although these cantilevers are schematically illustrated with only two extrusion layers and a few extrusion passes for simplicity, one of ordinary skill in the art in view of the disclosure would understand that structures having many more extrusion layers and having widths corresponding to many more extrusion passes fall within the scope of embodiments described herein.

FIGS. 3C and 3D schematically depict different ellipsoid structures that can be extruded in some embodiments. Ellipsoid structure 34 of FIG. 3C includes an outer layer 35a and an inner layer 35b that are both deposited in a circumferential manner and have corresponding circumferential fiber alignment, in accordance with some embodiments. Ellipsoid structure 36 of FIG. 3D includes an outer layer 37a having a circumferential fiber alignment and an inner layer 37b having a longitudinal fiber alignment, in accordance with some embodiments.

FIG. 3E schematically depicts a tube 38 including an outer layer 39a with a circumferential fiber alignment and an inner layer having a longitudinal fiber alignment. In other embodiments a tube may have circumferential fiber alignment in all layers.

As illustrated, in some embodiments, different extrusion layers or different portions of layers may have different preferential alignment directions which can be controlled based on the pattern of extrusion employed when extruding the structure. Further, one of ordinary skill in the art would understand that a structure could including multiple extrusion layers having one fiber alignment interspersed amongst other extrusion layers having a different fiber alignment. In some embodiments, a same extrusion layer could have one or more portions having one alignment and one or more other portions having a different alignment. One of ordinary skill in the art, in view of the present disclosure, will recognize that preferential fiber alignments are not limited to linear and circumferential. For example, preferential fiber alignments may be radial, spiral or follow some other pattern or combination of orientations.

As explained above, in some embodiments, at least some of the plurality of the polymeric fibers anisotropically align along a direction of extrusion in the three-dimensional structure of the fiber material. In some embodiments, one or more of an inner diameter of the nozzle, an average length of a polymeric fiber in the plurality of polymeric fibers, a maximum length of a polymeric fiber in the plurality of polymeric fibers, a ratio of weight of polymeric fibers to weight of carrier in the bioink material, and a composition of the plurality of polymeric fibers are selected such that at least some of the plurality of polymeric fibers anisotropically align along a direction of extrusion in the three-dimensional structure of the fiber material.

In some embodiments, an average length of the polymeric fibers is such that at least some of the polymeric fibers have an anisotropic distribution in the bioink upon extrusion. Specifically, an average length of the polymeric fibers is selected such that shearing forces exerted on the polymeric fibers during extrusion result in preferential alignment of at least some of the polymeric fibers along an extrusion direction in the resulting three-dimensional article. Examples of ranges of average lengths of fibers that result in preferential anisotropic alignment are described below and with respect to Example 4 below.

A maximum length of the polymeric fibers must be such that the fibers do not tangle during extrusion. The inventors determined that for some bioink materials and processing conditions, polymeric fibers longer than about 0.5 mm tend to tangle and clog the print nozzle if the extrusion nozzle inner diameter is smaller than or about equal to the polymeric fiber length. See discussion of Example 4 below. However, surprisingly, in similar bioinks where the longest polymeric fibers were shorter than about 0.5 mm, polymeric fibers with length longer than the inner diameter of the print nozzle did not clog or become entangled.

In some embodiments, fibers having shorter fiber lengths (e.g., fibers shorter than about 0.05 mm for some polymeric fibers and carriers and fibers shorter than about 0.03 mm for some polymeric fibers and carrier) do not significantly align in the extrusion direction, however, polymeric fibers having these shorter fiber lengths significantly contribute to post-extrusion crosslinking to achieve an interconnected fibrous scaffold in some embodiments. See discussion of Example 4 below.

In some embodiments, a mixture of polymeric fibers having shorter lengths (e.g., less than 0.03 mm or less than 0.05 mm) and longer lengths (e.g., between 0.05 mm and 0.5 mm) provide for anisotropic preferential alignment along the extrusion direction as well as sufficient isotropically oriented shorter fibers to form connections between the aligned fibers during cross-linking. See discussion of Example 4 below.

In some embodiments, each of the plurality of polymeric fibers has a length of less than 3 mm. In some embodiments, each of the plurality of polymeric fibers has a length of less than 0.5 mm. In some embodiments where at least some of the polymeric fibers have a length of more than 0.5 mm, a maximum length of the polymeric fibers is less than an inner diameter of the extrusion nozzle.

In some embodiments, an average length of a polymeric fiber in the plurality of polymeric fibers is in a range of 0.05 mm and 0.3 mm. In some embodiments, an average length of a polymeric fiber in the plurality of polymeric fibers is in a range of 0.07 mm to 0.25 mm. In some embodiments, an average length of a polymeric fiber in the plurality of polymeric fibers is in a range of 0.9 mm and 2.2 mm. In some of these embodiments, the plurality of polymeric fibers includes polymeric fibers having lengths less than 0.05 mm or lengths less than 0.03 mm.

In some embodiments, a length of each of the plurality of polymeric fibers is less than 0.25 mm, an average length of a polymeric fiber in the plurality of polymeric fibers is between 0.05 mm and 0.25 mm, and the plurality of polymeric fibers includes polymeric fibers having lengths less than 0.05 mm. In some of these embodiments, an average length of a polymeric fiber in the plurality of polymeric fibers is in a range of 0.15 mm to 0.25 mm. In some of these embodiments, an average length of a polymeric fiber in the plurality of polymeric fibers is in a range of 0.18 to 0.22 mm. In some of these embodiments, a ratio of a weight of the polymeric fibers to a weight of the carrier in the bioink material is less than 20. See discussion of Example 4 below.

In some embodiments, a length of each of the plurality of polymeric fibers is less than 0.25 mm, an average length of a polymeric fiber in the plurality of polymeric fibers is between 0.05 mm and 0.25 mm, and the plurality of polymeric fibers includes polymeric fibers having lengths less than 0.03 mm. In some of these embodiments, an average length of a polymeric fiber in the plurality of polymeric fibers is in a range of 0.05 mm and 1.5 mm. In some of these embodiments, an average length of a polymeric fiber in the plurality of polymeric fibers is in a range of 0.8 mm to 1.2 mm. In some of these embodiments, a ratio of a weight of the polymeric fibers to a weight of the carrier in the bioink material is less than 20. See discussion of Example 4 below.

In some embodiments, an inner diameter of the nozzle is in a range of 0.4 mm to 2.5 mm, a maximum length of a polymeric fiber in the plurality of polymeric fibers is greater than 0.25 mm and smaller than the inner diameter of the nozzle, the plurality of polymeric fibers includes polymeric fibers having lengths less than 0.05 mm or having lengths less than 0.03 mm, and a ratio of a weight of the polymeric fibers to a weight of the carrier in the bioink material is less than one. See discussion of Example 4 below.

In some embodiments, a length/diameter aspect ratio of each fiber in the plurality of fibers is between about 10:1 and about 200:1

Some embodiments are or include a method of making or providing a bioink material. In some embodiments, the method of forming a three-dimensional tissue scaffold includes providing a bioink material.

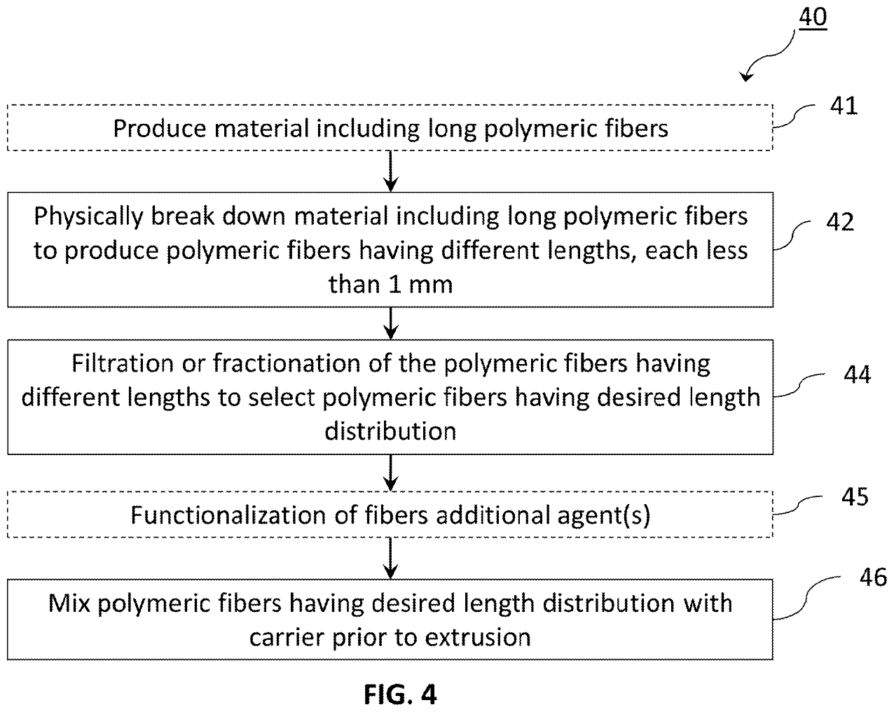

FIG. 4 is a flow chart schematically depicting a method 40 of making or providing a bioink material. In some embodiments the method 40 includes producing a material including long polymeric fibers (41), which is discussed further below. In some embodiments, the material including long polymeric fibers is provided or obtained and the method does not include providing the material including long polymeric fibers. Because this step is not included in some embodiments, the corresponding box 41 in the flow chart is shown with dashed lines.

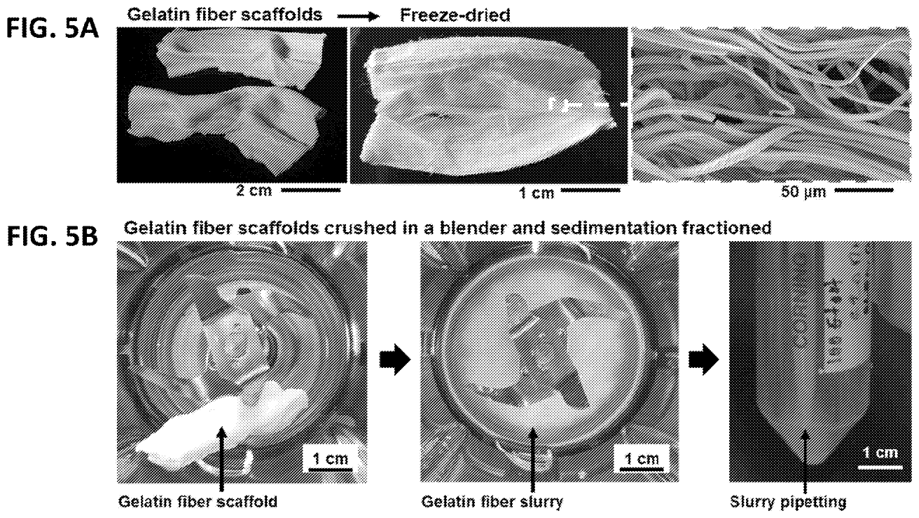

The material including long polymeric fibers is physically broken down to produce polymeric fibers having different lengths, each less than 1 mm (42). In some embodiments, this includes cutting the material into smaller pieces (e.g., about 1 mm in length) followed by crushing, grinding or both to reduce average fiber length. In some embodiments, the material including long polymeric fibers is freeze-dried prior to the cutting, crushing and grinding. In some embodiments, the polymeric fibers are mixed with a solvent prior to grinding. See also discussion of Example 1 and FIGS. 5A and 5B, below. In some embodiments, grinding conditions, such as applied force and grinding time, can be used to influence a length distribution of the fibers having different lengths resulting from the grinding.

The polymeric fibers having different lengths are filtered or fractionated to select polymeric fibers having a desired length distribution (44). The desired length distribution will include fibers that are all less than a selected maximum fiber length. In some embodiments, the desired length distribution includes an average fiber length falling within a selected range. In some embodiments, the fibers having different lengths are in a slurry for the filtering and fractionation. In some embodiments, the filtration and/or fractionation includes sedimentation, centrifugation, passage through porous material filters and/or other known methods. See also discussion of Example 1 and FIG. 5B below.

In some embodiments, the polymeric fibers are functionalized with additional agents (45). The box for this step is indicated using dashed lines as the step is not present in some embodiments. In some embodiments, some or all of the functionalization occurs after the desired length distribution of fibers is achieved. In some embodiments, some or all of the functionalization occurs during formation of the long polymeric fibers. In some embodiments, some or all of the functionalization occurs after the polymeric fibers are physically broken down, but before the filtration or fractionation. In some embodiments, the carrier includes one or more additional agents.

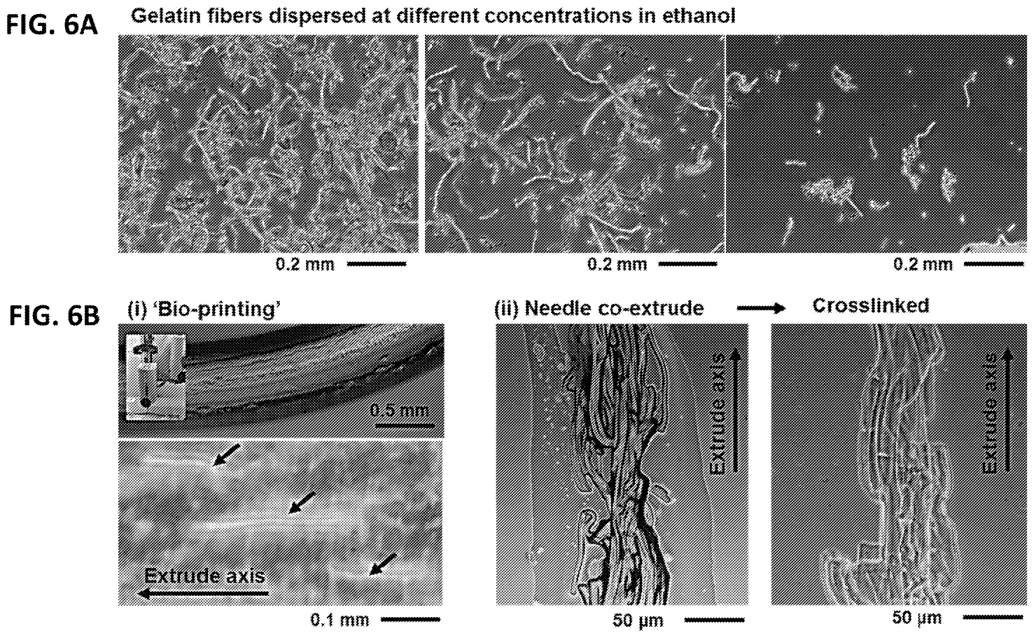

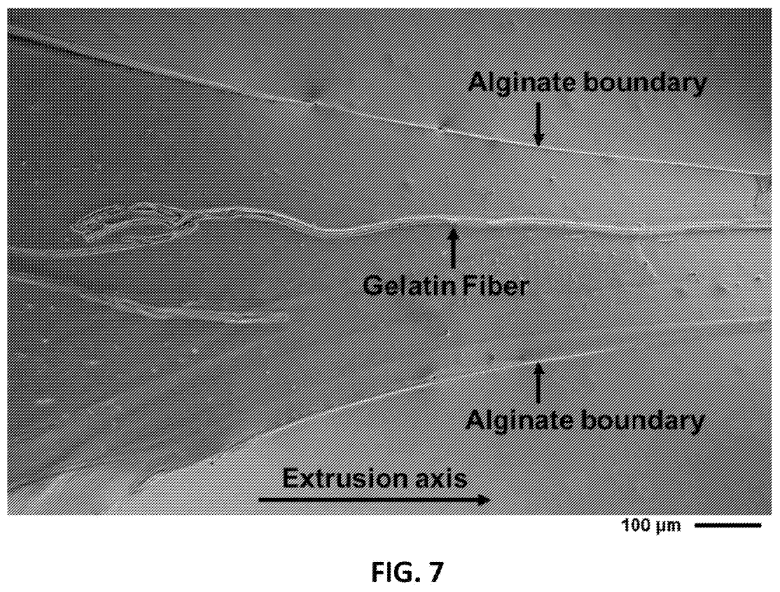

The method includes mixing the polymeric fibers having a desired length distribution with a carrier prior to extrusion (46). In some embodiments, the polymeric fibers having the desired length distribution are in a liquid after grinding, and the polymeric fibers are dried to evaporate the liquid prior to being mixed with the carrier. See also discussion of Example 1 FIG. 6A below. In some embodiments, the dry polymeric fibers are suspended in water and then the polymeric fibers suspended in water are mixed with the carrier to form the bioink material. In some embodiments, the polymeric fibers have a tendency to dissolve in the carrier material and so the mixing with the carrier occurs shortly before extrusion of the bioink material. For example, in some embodiments employing pure uncrosslinked gelatin polymeric fibers, the polymeric fibers should be mixed with the carrier material less than about 30 minutes prior to cross-linking. In some embodiments, polymeric fibers that have a tendency to dissolve in the carrier can be radiation hardened by exposure to electromagnetic radiation to increase the stability of the polymeric fibers in the carrier. For example, radiation hardening of pure gelatin polymeric fibers may enable the fibers to be mixed with the carrier material up to 24 to 48 hours prior to cross-linking.

Some embodiments provide kits for bioink materials to be used with a 3-D printing or additive manufacturing system. A kit includes a plurality of biocompatible polymers and a carrier material in some embodiments. The plurality of polymeric fibers have an average length in a range of 0.07 mm to 0.25 mm, and each polymeric fiber has a diameter in a range of 0.05 mm and 0.3 mm and comprises one or more biocompatible polymers in some embodiments. The carrier comprises a hydrogel forming solution in some embodiments. The average length of the plurality of polymeric fibers results in at least some of the polymeric fibers being preferentially oriented along an extrusion direction when the bioink is extruded from a three-dimensional printer or additive manufacturing system.

In some embodiments, mixing the plurality of biocompatible polymers and the carrier material produces the bioink material. In some embodiments, water is added to the plurality of biocompatible polymers, the carrier material, or both prior to mixing to produce the bioink material.

In some embodiments, each of the plurality of polymeric fibers has a length of less than 3 mm. In some embodiments, each of the plurality of polymeric fibers has an average length in a range of 0.05 mm to 0.3 mm. In some embodiments, each of the plurality of polymeric fibers has an average length in a range of 0.9 mm and 2.2 mm. In some embodiments, the plurality of polymeric fibers includes polymeric fibers having lengths less than 0.05 mm. In some embodiments, the plurality of polymeric fibers includes polymeric fibers having lengths less than 0.03 mm. In some embodiments, the bioink material produced using the kit is extrudable at ambient temperature. In some embodiments, the carrier comprises one or more of alginate or gelatin. In some embodiments, the one or more biocompatible polymers include one or more of gelatin, hyaluronic acid, and polycaprolactone.

In some embodiments, the kits may also include an agent to be added to a bath for interacting with the carrier to form a gel upon extruding the bioink material into the bath.

Embodiments also include the bioink material themselves.

### Polymers for Fibers

Suitable polymers for use in the polymeric fibers of the methods, kits, and compositions of the invention include biocompatible synthetic and biogenic polymers, such as polymers that promote cell attachment and 3D tissue culture, polymers that provide structural or functional support to engineered tissues, and edible polymers, and combinations thereof. Examples described herein employed fibers including gelatin, hyaluronic acid (HA), and polycaprolactone (PCL). These fibers were chosen to highlight the use of polymer (PCL) and biopolymer materials. Other examples of suitable biocompatible polymers are listed below.

Suitable synthetic polymers include, for example, poly(urethanes), poly(siloxanes) or silicones, poly(ethylene), poly(vinyl pyrrolidone), poly(2-hydroxy ethyl methacrylate), poly(N-vinyl pyrrolidone), poly(methyl methacrylate), poly(vinyl alcohol), poly(acrylic acid), polyacrylamide, poly(ethylene-co-vinyl acetate), poly(ethylene glycol), poly(methacrylic acid), polylactides (PLA), polyglycolides (PGA), poly(lactide-co-glycolides) (PLGA), polyanhydrides, polyphosphazenes, polygermanes, polyorthoesters, polyesters, polyamides, polyolefins, polycarbonates, polyaramides, polyimides, polycaprolactone (PCL), and copolymers and derivatives thereof, and combinations thereof.

Suitable biogenic polymers (or bio-derived polymers), e.g., proteins, polysaccharides, lipids, nucleic acids or combinations thereof, include, but are not limited to, silk (e.g., fibroin, sericin, etc.), keratins (e.g., alpha-keratin which is the main protein component of hair, horns and nails, beta-keratin which is the main protein component of scales and claws, etc.), elastins (e.g., tropoelastin, etc.), fibrillin (e.g., fibrillin-1 which is the main component of microfibrils, fibrillin-2 which is a component in elastogenesis, fibrillin-3 which is found in the brain, fibrillin-4 which is a component in elastogenesis, etc.), fibrinogen/fibrins/thrombin (e.g., fibrinogen which is converted to fibrin by thrombin during wound healing), fibronectin, laminin, collagens (e.g., collagen I which is found in skin, tendons and bones, collagen II which is found in cartilage, collagen III which is found in connective tissue, collagen IV which is found in vascular basement membrane, collagen V which is found in hair, etc.), collagen VI which is found in pancreatic islets and adipose, vimentin, neurofilaments (e.g., light chain neurofilaments NF-L, medium chain neurofilaments NF-M, heavy chain neurofilaments NF-H, etc.), proteoglycans, integrins, amyloids (e.g., alpha-amyloid, beta-amyloid, etc.), actin, myosins (e.g., myosin I-XVII, etc.), titin which is the largest known protein (also known as connectin), gelatin, alginate, chitin which is a major component of arthropod exoskeletons, hyaluronic acid which is found in extracellular space and cartilage (e.g., D-glucuronic acid which is a component of hyaluronic acid, D-N-acetylglucosamine which is a component of hyaluronic acid, etc.), etc., and combinations thereof. Exemplary biogenic polymers, for use in the present invention include, but are not limited to carbohydrate polymers found in the body e.g., glycosaminoglycans (GAGs), heparan sulfate found in extracellular matrix, chondroitin sulfate (e.g., chondroitin 4-sulfate, chondroitin 6-sulfate) which contributes to tendon and ligament strength, keratin sulfate which is found in extracellular matrix, etc. In some embodiments, biocompatible polymers for fibers also include chitosan, starches, sugars, polysaccharides, or combinations thereof.

### Carrier Materials

In some embodiments the carrier includes material that forms a hydrogel or a hydrogel forming solution. In some embodiments, the carrier includes alginate. In some embodiments, the carrier includes gelatin. In some embodiments the carrier includes pectin. In some embodiments, the carrier includes hydrogel forming solution or a material that forms a hydrogel based on proteins (e.g., alginate, gelatin, pectin, collagen, elastin, fibrin, MATRIGEL, decellularized matrix, matrix produced by cell culture, etc.), a hydrogel based on polysaccharides (e.g., HA, alginate, chitosan, dextran, etc.), a synthetic hydrogel (e.g., Poly(ethylene glycol) (PEG), Poly(vinyl alcohol) (PVA), Poly(acrylic acid) (PAA), poly(lactic acid) (PLA), etc.), or any combination of the aforementioned.

### Gelation

In some embodiments, the bioink is extruded into a bath and the bath includes one or more agents to gel the carrier after extrusion into the bath. For example, gelation of a carrier including an alginate solution can be by a bath including divalent cations, such as Ca²⁺ in diH₂O).

In some embodiments, the bioink is extruded into a bath having a different temperature than that of the bioink and a temperature change in the bioink after extrusion in the bath causes gelation of the carrier. For example, for a carrier including a gelatin solution, the carrier is prepared and used at an elevated temperature e.g., 40° C., 50° C. When extruded into a cooled bath or a temperature controlled print substrate at a temperature less than the gelation temperature (T<T_gel), which is also known as the sol-gel transition temperature, a degree of gelation of the gelatin depends on the temperature of the bath or the temperature controlled substrate. The sol-gel transition temperature (T_gel) depends on the concentration of gelatin as different concentrations of gelatin will have different sol-gel transition temperatures. For example a 5% (w/s) solution of gelatin in diH₂O gels when T<˜5-10° C., a 10% solution of gelatin gels when T<˜5-20° C., and a 20% solution of gelatin gels when T<˜30° C. The embedded fibers should have a sol-gel transition temperature greater than the carrier, which enables carrier to be selectively removed after crosslinking the embedded fibers by heating the extruded structure.

As another example of thermal gelation, Pluronics F127 (poloxamer 407) is a commonly used thermo-responsive sacrificial ink, with concentration-dependent sol-gel transition between ˜10° C. and 40° C. However, unlike gelatin, pluronics F127 is a liquid at lower temperatures and a gel at higher temperatures. Thus, the bioink is extruded onto a heated substrate or into a heated bath (T˜10° C. to 40° C., depending on pluronics concentration, for example 40% pluronics F127 w/w in diH2O has a ˜40° C. sol-gel temperature). After crosslinking of the fibers, the object is refrigerated to remove the pluronics.

### Cross-Linking and Heat Fusing

In some embodiments, a cross-linking agent comprises (N-(3-dimethylaminopropyl)-N′-ethylcarbodiimide hydrochloride) (EDAC or EDC hydrochloride). In some embodiments, a cross-linking agent comprises microbial transglutaminase (mTG). In some embodiments, a cross-linking agent comprises genipin. In some embodiments, a cross-linking agent comprises riboflavin. A cross-linking agent includes calcium chloride.

As noted above, in some embodiments, the cross-linking of the polymeric fibers in the extruded bioink material is chemical or enzymatic. For example, gelatin and hyaluronic acid can be chemically crosslinked using EDAC or a combination of EDC and N-hydroxysuccinimide (NHA). In another example, genipin can be used to crosslink chitosan fibers or gelatin fibers. Riboflavin in combination with UV radiation can be used to crosslink gelatin fibers, in accordance with some embodiments. In some embodiments, citric acid can be used to crosslink starch fibers or gelatin fibers.

In some embodiments, the crosslinking of the polymeric fibers is enzymatic. For example, microbial transglutaminase (mTG) can be used to cross-link gelatin. Various gelatin concentrations (˜1-10%) and various mTG concentrations (2 U/gram to 40 U/gram) all produce gels, and the resulting gels have mechanical properties (elastic modulus) that depend on gel and mTG concentrations. Because the cross-linking can take more than one hour, gelatin fibers should stabilized (e.g., UV-stabilized) prior to enzymatic cross-linking that takes place in aqueous solutions.

Some polymeric fibers (e.g., protein-based fibers such as gelatin fibers and hyaluronic acid fibers) can be UV-stabilized prior to immersion in the carrier for printing or prior to immersion in aqueous solutions for enzymatic crosslinking. This is not a stable crosslinking compared to chemical or enzymatic methods: fibers will still degrade in aqueous solutions. This can be useful to stabilize fibers for (i) immersion in carriers, or (ii) for enzymatic crosslinking that occurs in aqueous solutions. Gelatin fibers hardened by exposure to UV radiation for extended time periods (48 hours) resisted degradation in aqueous solutions, allowing them to be immersed in aqueous carrier inks such as alginate for up to 1 day without significant change in morphologies: i.e., UV-stabilization increases the time fibers can be in carrier inks during printing and subsequent crosslinking.

UV-stabilization of gelatin fibers also supports the use of a bioink material including gelatin fibers in a gelatin solution carrier, where sol-gel transition temperatures differ for fiber or carrier components: In these cases, 3D structures are produced by extruding the bioink material with nozzle temperature higher than the carrier sol-gel temperature onto or in to substrates with temperature below the carrier sol-gel. The resulting 3D objects are then kept at a temperature slightly above the carrier sol-gel transition such that fibers can be crosslinked selectively while the carrier degrades where carrier degradation rate by temperature is greater than the carrier crosslinking rate and the fiber crosslinking rate is greater than the fiber degradation rate by temperature.

In some embodiments, heat fusing is used instead of or along with crosslinking to connect the polymeric fibers in the extruded material. PCL fibers are fused by heat treatment, which is not crosslinking per se. Polymeric fibers such as polycaprolactone (PCL) can be fused by heat treatments. After printing a 3D structure of a bioink containing embedded PCL fibers, exposure to temperatures >60° C. causes PCL fibers to fuse. In one embodiment, a 3D printed object consisting of alginate with embedded PCL fiber network is immersed in a heated water bath (T=60° C., or 70, 80, 90° C.), for times ranging from 1 minute, 10 minutes, 30 minutes, 1 hour, 6 hours, 24 hours, depending on fiber density and the degree of fusing required. The melting point of PCL is 60° C. In another embodiment, the extruded 3D bioink structure is immersed in water within tubing or jars and autoclaved (T˜115° C. for 30 minutes to 1 hour). During the heat fusing, the carrier material (e.g. alginate) continues its role as a structural stabilizer of the embedded fiber network as the fibers are melted and fuse together. Upon cooling, the network is connected, and the carrier may be removed.

This temperature-based strategy is expected to work for a broad range of polymeric fibers: As long as the carrier does not melt (e.g., alginate is stable during autoclaving or heat baths not containing salts), then embedded fibers may be fused by melting/cooling.

Preparation of Long Polymeric Fibers or Material including Long Polymeric Fibers

Any suitable method may be used to prepare long polymeric fibers or a material including long polymeric fibers (e.g., a non-woven polymeric fiber sheet) that is processed to produce polymeric fibers having a desired length distribution that are disposed it the carrier to form the bioink. Suitable methods include, but are not limited to rotary jet spinning, immersion rotary jet spinning, pull-spinning, electrospinning, solution blow spinning, melt extrusion, microfluidic extrusion, etc. These methods can be used to produce fibers with diameters in a range of 0.1 μm to 20 μm and lengths typically exceeding 1 cm.

In one embodiment, long polymeric fibers or a material including long polymeric fibers (e.g., a non-woven polymeric fiber sheet) are/is formed by ejecting a polymer solution from a reservoir onto a collector (e.g., a stationary collector, a rotating mandrel or mandrel assembly). In some embodiments, rotary jet spinning (RJS) is used to create long polymeric fibers that are collected into non-woven polymeric fiber sheets. Suitable RJS devices and uses of the devices for fabricating the long polymeric fibers and non-woven polymeric fiber sheets are described in U.S. Patent Publication No. 2012/0135448, U.S. Patent Publication No. 2013/0312638, U.S. Patent Publication No. 2014/0322515, the entire contents of each of which are incorporated in their entirety by reference.

In some embodiments, immersion rotary jet pinning (iRJS) is used to create long polymeric fibers as described in U.S. Patent Publication No. 2015/0354094, the entire content of which is incorporated by reference in its entirety.

In other exemplary embodiments, the polymeric fibers may be flung using a pull spinning technique onto a collector (e.g., a stationary collector, a rotating mandrel or mandrel assembly). Suitable pull spinning devices and uses of the devices for fabricating the non-woven polymeric fiber sheets are described in U.S. Patent Publication No. 2014/0322515, the entire contents of which is incorporated in its entirety by reference.

### III. Uses of Tissue Scaffolds of the Invention

As noted above, the fibers and/or carrier and/or scaffolds may also include one or more additional agents, e.g., a plurality of living cells, e.g., muscle cells, neuron cells, endothelial cells, and epithelial cells; biologically active agents, e.g., lipophilic agents, peptides, lipids, nucleotides, small molecules; fluorescent molecules, metals, ceramics, nanoparticles, and pharmaceutically active agents.

The one or more additional agents may be added to a polymer solution used to fabricate the fibers (e.g., enabling the agent to be incorporated into the fibers themselves); one or more fibers may be coated (e.g., fully or partially) with one or more additional agents prior to being combined with the carrier, the carrier may include one or more additional agents; and/or a scaffold may be coated (e.g., fully or partially) with one or more additional agents.

For example, by including one or more additional agents within the fibers as they are produced, e.g., in the solution of the polymer used to produce the fibers, including them after production of the fibers, and/or including them in the carrier, and forming a cross-linked scaffold including the one or more additional agents, ECM-inspired nanofibrous scaffolds with controlled-release of cell-instructive factors may promote beneficial immune system reactions and endogenous repair mechanisms within regenerative medicine applications. They may promote cell programming, cell reprogramming, and tissue genesis within broader tissue engineering applications (e.g., in vitro disease models). In some embodiments, additional agents for cell programming include the so-called “Yamanaka factors” (transcription factors Oct4, Sox2, cMyc, and Klf4), which are used to induce pluripotency in somatic cells.

Embodiments also include engineered tissues formed using the three-dimensional tissue scaffolds.

Another use of the three-dimensional tissue scaffolds in some embodiments is the delivery of one or more substances to a desired location and/or in a controlled manner. In some embodiments, the tissue scaffold is used to deliver the materials, e.g., a pharmaceutically active substance. In other embodiments, the tissue scaffold is used to deliver substances that are contained in the polymeric fibers or that are produced or released by substances contained in the polymeric fibers materials. For example, polymeric fibers containing cells can be implanted in a body and used to deliver molecules produced by the cells after implantation. The present compositions can be used to deliver substances to an in vivo location, an in vitro location, or other locations.

The ability to seed the tissue scaffolds of some embodiments with living cells also provides the ability to build tissue, organs, or organ-like tissues. Cells included in such tissues or organs can include cells that serve a function of delivering a substance, seeded cells that will provide the beginnings of replacement tissue, or both.

In some embodiments of the invention, a tissue scaffold is treated with a plurality of living cells and cultured under appropriate conditions to produce a bioengineered tissue.

In some embodiments, the tissue scaffolds contacted or seeded with living cells incorporate a drug such that the function of the implant will improve. For example, antibiotics, anti-inflammatories, local anesthetics or combinations thereof, can be incorporated into the tissue scaffold for a bioengineered organ to speed the healing process.

Examples of bioengineered tissue include, but are not limited to, bone, dental structures, joints, cartilage, (including, but not limited to articular cartilage), skeletal muscle, smooth muscle, cardiac muscle, tendons, menisci, ligaments, blood vessels, stents, heart valves, corneas, ear drums, nerve guides, tissue or organ patches or sealants, a filler for missing tissues, sheets for cosmetic repairs, skin (sheets with cells added to make a skin equivalent), soft tissue structures of the throat such as trachea, epiglottis, and vocal cords, other cartilaginous structures such as articular cartilage, nasal cartilage, tarsal plates, tracheal rings, thyroid cartilage, and arytenoid cartilage, connective tissue, vascular grafts and components thereof, and sheets for topical applications, and repair of organs such as livers, kidneys, lungs, intestines, pancreas visual system, auditory system, nervous system, and musculo skeletal system.

In one particular embodiment, a tissue scaffold is contacted with a plurality of living muscle cells and cultured under appropriate conditions to guide cell growth with desired anisotropy to produce a muscle thin film (MTF) or a plurality of MTFs prepared as described in U.S. Patent Publication Nos. 20090317852 and 20120142556, and PCT Application No. PCT/US2012/068787. The entire contents of each of the foregoing are incorporated herein by reference.

Tissue scaffolds contacted with living cells can also be used to produce prosthetic organs or parts of organs. Mixing of committed cell lines in a three dimensional tissue scaffold can be used to produce structures that mimic complex organs. The ability to shape the tissue scaffold and control fiber anisotropy enables preparation of complex structures to replace organs such as liver lobes, pancreas, other endocrine glands, and kidneys. In such cases, cells are implanted to assume the function of the cells in the organs. Preferably, autologous cells or stem cells are used to minimize the possibility of immune rejection.

In some embodiments, tissue scaffolds contacted with living cells are used to prepare partial replacements or augmentations. For example, in certain disease states, organs are scarred to the point of being dysfunctional. A classic example is hepatic cirrhosis. In cirrhosis, normal hepatocytes are trapped in fibrous bands of scar tissue. In one embodiment, the liver is biopsied, viable liver cells are obtained, cultured in the tissue scaffold, and re-implanted in the patient as a bridge to or replacement for routine liver transplantations.

In another example, by growing glucagon secreting cells, insulin secreting cells, somatostatin secreting cells, and/or pancreatic polypeptide secreting cells, or combinations thereof, in separate cultures, and then mixing them together with a tissue scaffold, an artificial pancreatic islet is created. One or more artificial pancreatic islets are then placed under the skin, retroperitoneally, intrahepatically or in other desirable locations, as implantable, long-term treatments for diabetes.

In other examples, hormone-producing cells are used, for example, to replace anterior pituitary cells to affect synthesis and secretion of growth hormone secretion, luteinizing hormone, follicle stimulating hormone, prolactin and thyroid stimulating hormone, among others. Gonadal cells, such as Leydig cells and follicular cells are employed to supplement testosterone or estrogen levels. Specially designed combinations are useful in hormone replacement therapy in post and perimenopausal women, or in men following decline in endogenous testosterone secretion. Dopamine-producing neurons are used and implanted in a matrix to supplement defective or damaged dopamine cells in the substantia nigra. In some embodiments, stem cells from the recipient or a donor can be mixed with slightly damaged cells, for example pancreatic islet cells, or hepatocytes, and placed on a tissue scaffold and later harvested to control the differentiation of the stem cells into a desired cell type. In other embodiments thyroid cells can be seeded and grown to form small thyroid hormone secreting structures. This procedure is performed in vitro or in vivo. The newly formed differentiated cells may be introduced into the patient.

### IV. EXAMPLES

### Example 1. Gelatin Fiber Bioink Material and Anisotropy in Extruded Ink

A material including long gelatin fibers was processed to form a mix of gelatin fibers having a desired length distribution. The gelatin fibers were produced by immersion rotary jet spinning of 20% gelatin solutions at 40° C. into ethanol/water precipitation baths. See explanation of iRJS in U.S. Patent Publication No. 2015/0354094, which is incorporated by reference herein in its entirety. FIG. 5A includes images of a gelatin fiber material that was freeze dried and cut prior to being physically broken down. The gelatin fiber material included gelatin fibers having diameters in a range of about 2 μm to about 20 μm. The gelatin fiber material was produced using an immersion rotary jet spinning (iRJS) device. A gelatin solution was extruded from 0.5 mm orifices in the rotating reservoir walls of the iRJS device into an ethanol/water precipitation bath with the reservoir rotating at a fixed rotation rate of 15,000 RPM. Gelatin solutions were fed into a reservoir at a rate of 10 mL/min and produced gelatin fibers material at a rate of ˜100 g/hr, dry weight. A circulating precipitation bath vortex was maintained during spinning with a rotating collector fixture.

Using pure gelatin precursor solutions (20% w/w porcine Type 300A), fibrillar gelatin was obtained when the ethanol concentration was 70% or higher. Bath water concentrations higher than 30% led to fiber fusion and partial dissolution in the bath during a 5-minute spin time. Replicate samples were spun for three bath conditions (ethanol:water=100:0, 80:20, 70:30), with average fiber diameter decreasing with increasing bath ethanol concertation; pure ethanol produced gelatin fibers with average diameters of about 15 microns, and 70:30 ethanol:water produced gelatin fibers with average diameter of about 3 microns.

FIG. 5B includes images of the gelatin fiber material being crushed and ground in a commercial blender when mixed with ethanol to reduce average fiber length producing a slurry including polymeric fibers having different lengths, each less than 1 mm. The slurry was then filtered and fractionated to obtain a desired length distribution for the gelatin fibers.

FIG. 6A includes microscope images of the gelatin fibers having the desired length distribution dispersed in ethanol in three different concentrations showing the different lengths of the fibers. From left to right, the concentrations of fibers in solvent were 100 mg/mL, 50 mg/ML and 10 mg/mL.

The gelatin fibers were dried and then suspended in an alginate carrier for extrusion. Sodium alginate (E401, PebChem ID:5102882, CAS: 14984-39-5) was mixed in deionized water (diH₂O) to form the carrier. The optimal concentration of alginate used as a carrier gel depends on the alginate molecular weight. For low-viscosity alginate (Sigma A1112, MW=216.12 g/mol), successful printing was achieved using concentrations between 5% w/w and 15% w/w alginate mixed in diH₂O. For a high-viscosity alginate (Modernist pantry, sodium alginate, Id:1007-50, MW unknown), successful printing was achieved using concentrations between 1% and 3% w/w alginate mixed in diH₂O.

Bioink materials including different relative concentrations of gelatin fibers and an alginate solution carrier (a 1% solution of a high viscosity sodium alginate from Modernist Pantry, ID:1007-50 with MW unknown in diH₂O) were extruded in lines onto slides for imaging. Successful printing was achieved using alginate solution concentrations of 1% to 3% w/w of alginate in diH₂O. FIG. 6B includes (i) a microscope image (with bioprinter insert) of a bioink material extruded line that was gelled in a 5 minute immersion in 2.5% CaCl₂ in diH₂O to form a filament and a higher resolution microscope image of the gelled bioink material filament showing gelatin fibers (indicated with arrows) within the filament aligned with the extrusion axis at a relatively low fiber concentration of 10 mg/ml. FIG. 6B ii) includes a microscope image of a bioink material including a relatively high fiber concentration of gelatin fibers of 100 mg/ml extruded from a needle onto a slide without gelling of the alginate carrier and a microscope image after cross-linking of the gelatin fibers and removal of alginate carrier. The alginate carrier was removed during fiber cross-linking, which was by immersion in room temperature polybutylene succinate (PBS) containing 10 mM EDC and 4 mM NHS. For the relatively high fiber concentration, the fibers were also aligned preferentially along an extrusion axis.

The bioink material was extruded into a bath for gelation of the alginate, or the extruded bioink material was immersed in a bath for gelation after extrusion. Gelation of the alginate carrier was by a solution including divalent cations, in this example Ca²⁺ in diH₂O. In various embodiments, the concentration of divalent cations (e.g., Ca²⁺) for gelation of alginate may be about 0. 1%, about 0.5%, “about 1%, about 1.25%, about 1.5%, about 1.75%, about 2%, about 2.25%, about 2.5%, about 2.75%, about 3%, about 3.25%, about 3.5%, about 25 3.75%, about 4%, about 4.25%, about 4.5%, about 4.75%, or about 5% calcium chloride. Amounts intermediate to the above recited amount (e.g., about 2.3%) are also contemplated to be part of the invention.

FIG. 7 includes a microscope image of an extruded bioink material line including gelatin fibers in an alginate hydrogel carrier where the gelatin fibers have diameters on the order of about 10 μm and a length of about 1 mm in accordance with an example embodiment. The carrier was high viscosity alginate in a 1% w/w solution of diH₂O. Even with these very long fibers, the gelatin fibers were extended and aligned along the extrusion axis. In FIGS. 6A (ii) and 7, the bioink material was extruded through a needle and deposited in a straight line without subsequent gelation merely for illustrative and imaging purposes to verify preferential alignment of the gelatin fibers along an extrusion axis.

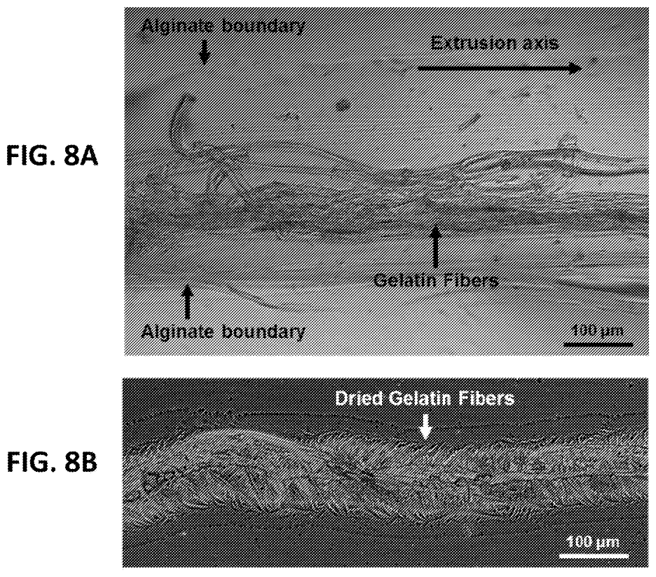

A gelatin fiber and alginate hydrogel carrier bioink material was produced with a high concentration of gelatin fibers of 100 mg/ml and extruded through a needle in a line without subsequent gelation merely for illustrative and imaging purposes. As shown in FIG. 8A, an extruded line of the bioink material shows high anisotropy of fibers and preferential alignment of fibers alone the extrusion axis. The line of bioink material was dried in a salt solution, specifically solution of PBS containing 8 g/L NaCl. FIG. 8B includes a microscope image of the extruded line after being dried in the solution containing salts where the salt crystallization sites are determined by gelatin fiber features, giving rise to regularly spaced salt dendrites sprouting from the gelatin fibers, demonstrating that that extrusion conditions determine fiber alignment, and subsequent chemical reactions guided by fibers in accordance with an example embodiment.

### Example 2. Printing of Bioink Material including Hyaluronic Acid (HA)

A bioink material was made from HA fibers and an alginate solution. The HA fibers were formed by iRJS of a 4% HA solution in ethanol/water precipitation baths. The HA fibers had diameters in a range of about 10 microns to about 30 microns with an average fiber diameter of 30 microns and a length distribution with a maximum fiber length of about 0.5 mm and an average fiber length of about 0.1 mm. The alginate solution included 5% low viscosity sodium alginate (MMW=216.12 g/mol, Sigma A112) in water. Different weight ratios of HA fibers and alginate solution were employed for bioink materials. The bioink materials were extruded using needles having varying gauges and a 3D printer.

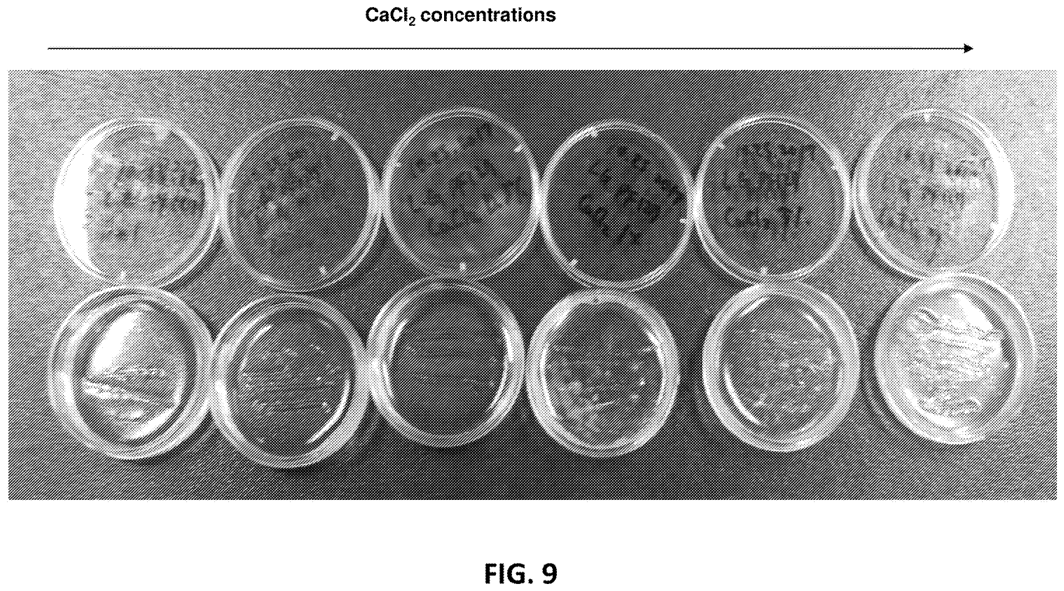

The Alg:HA bioink materials were extruded into a Pluronic F127 bath (poloxamer 407, 5% w/v in DI H₂O) having different concentrations of CaCl₂. By extruding the bioink materials into a bath containing CaCl₂, the alginate gels due to interaction with Ca²⁺ ions. FIG. 9 includes images of Alg:HA filaments formed by extruding the bioink into baths having different concentrations of CaCl₂. Using this bioink material composition (50 mg HA/1 mL DI H₂O mixed 1:1 with 5% sodium alginate (MMW, Sigma 2133)) and this needle inner diameter (0.21 mm), an optimal concentration of CaCl₂ in the bath was determined to be 3%.

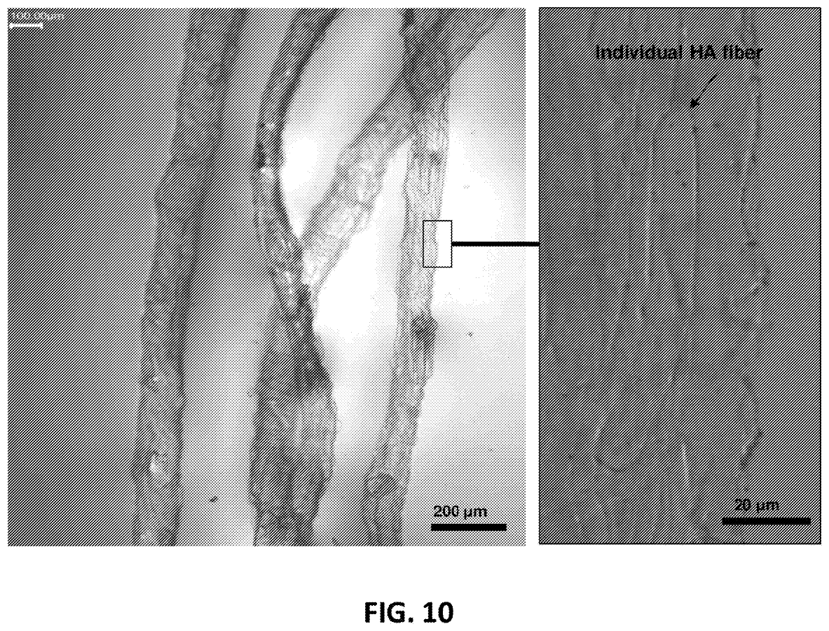

FIG. 10 includes microscope images of Alg;HA filaments formed by extrusion of the bioink material (50 mg HA/1 mL DI H₂O mixed 1:1 with 5% sodium alginate) using a 27 G needle (0.21 mm inner diameter) into a 3% CaCl₂ bath. As illustrated in the detail of FIG. 10, the individual HA fibers were preferentially aligned with the extrusion axis in the filaments.

### Example 3. 3D Printing of Structure using Hyaluronic Acid (HA) Fiber and Alginate Bioink

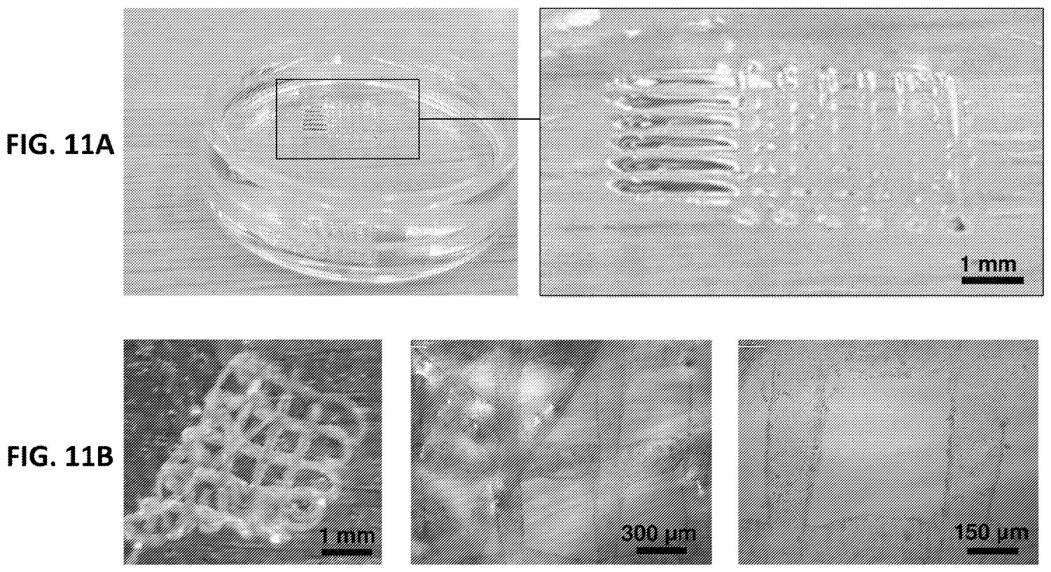

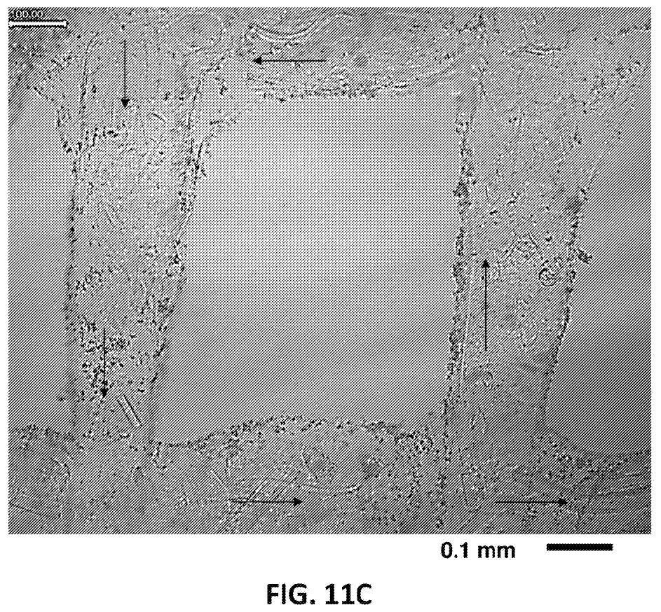

A mesh structure was 3D printed in a CaCl₂ bath using the HA and alginate bioink material described above. FIG. 11A shows the mesh structure as 3D printed in the bath. FIG. 11B shows the mesh structure after removal from the bath. Arrows showing an extrusion direction are in the higher magnification image of the mesh in FIG. 11C, which shows the preferential alignment of the HA fiber with the print direction.

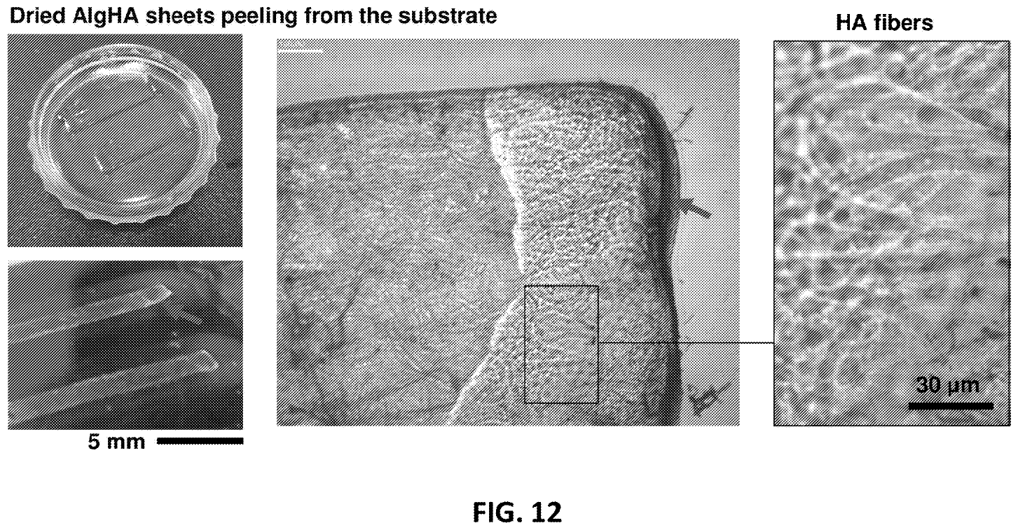

Sheets were formed by extruding a HA fiber and gelatin bioink having high concentration of HA fibers (50 mg HA in one milliliter of distilled water, mixed 1:1 with 5% sodium alginate (MMW, Sigma 2133) into a CaCl₂ bath using a 3D printer. Upon drying, the sheets peeled away from the substrate and showed anosotripic HA fiber orientation along the print direction as shown in the images of FIG. 12.

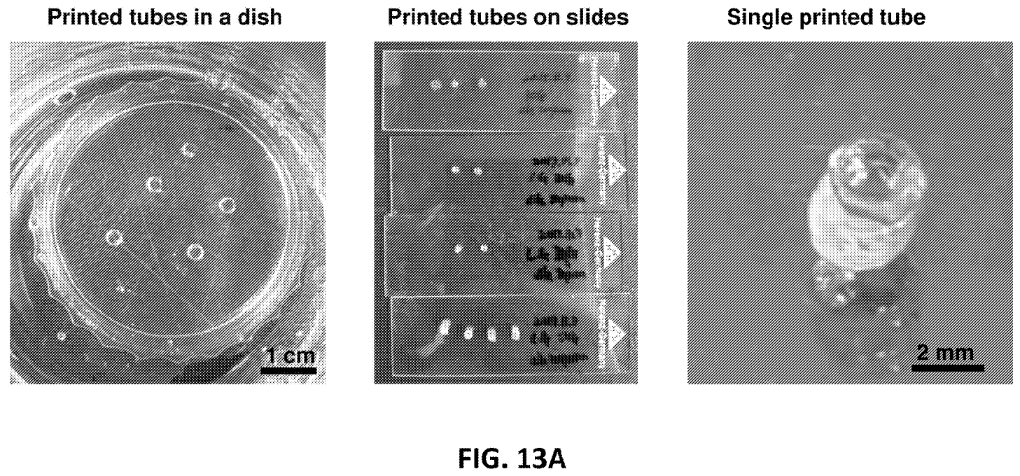

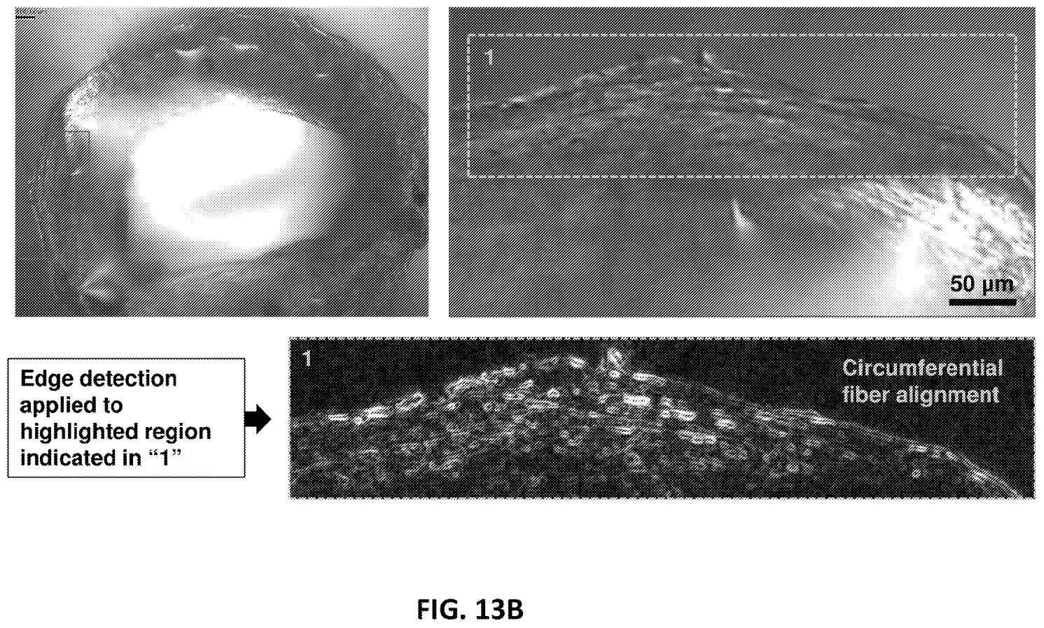

Freestanding tubes of a HA fiber and alginate carrier bioink material were printed using a 3D printer into petri dishes that contained pluronics F127 gels for support. After printing the whole petri dish was immersed in 3% CaCl2 and stored overnight in a refrigerator (4° C.). During overnight storage, the alginate was gelled by CaCl2 diffusion through the pluronics and the pluronics dissolves (at a slower rate than alginate gelation). Resulting freestanding tubes are shown in the images of FIG. 13A. It was determined that a 30 G needle having an inner diameter of about 0.159 mm and a pressure of 350 kPa and a 50 μm gap size in the z-direction provided the best results for tube printing using the HA fiber and gelatin bioink. The magnified images of a cross-section of one of the printed tubes in FIG. 13B show the circumferential alignment of the HA fibers along the print direction.

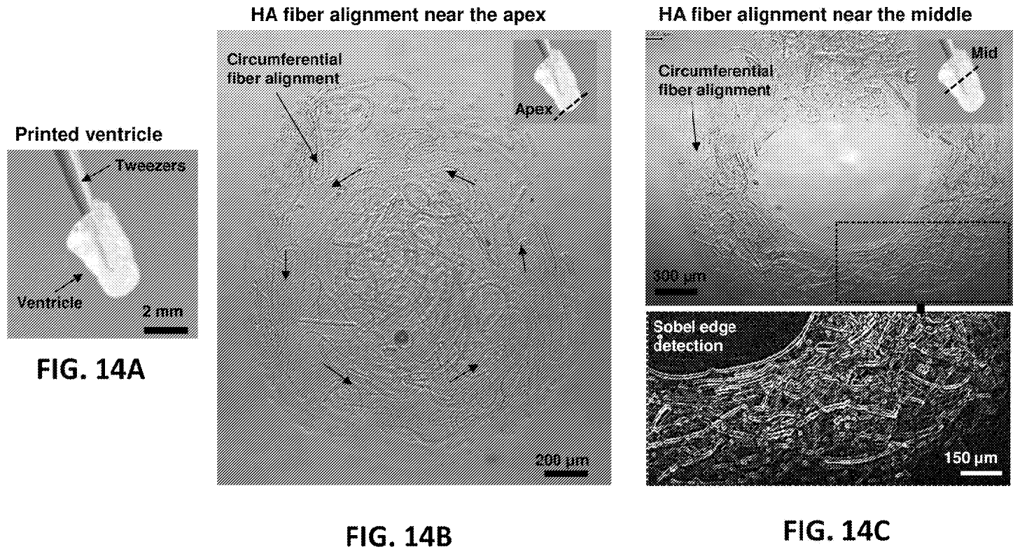

A 3D scale model of a ventricle was 3D printed using a HA fiber and alginate bioink using the same materials and processes as those describes above with respect to the freestanding tubes. For this model ventricle, HA fibers with lengths between 10 mm and 300 mm aligned with the print direction, but longer fibers were prone to entanglement. As shown in the cross-sectional images of FIG. 14A-14C, the HA fibers were aligned with the print direction throughout the wall of the model ventricle.

### Example 4. Determination of Fiber Length Distributions and 3D Printing Parameters for Successful Printing of 3D Scaffolds with Anisotropic Fiber Orientations Along the Print Direction

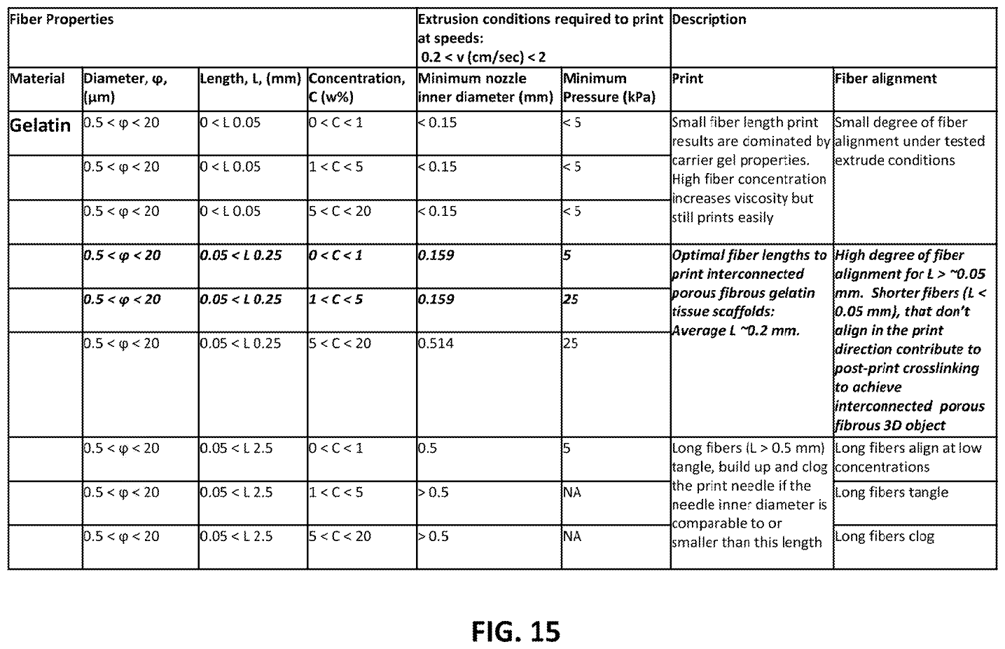

Various bioinks including different distributions of lengths of gelatin fibers in carriers including a hydrogel forming solution were evaluated to determine the effect of maximum fiber and average fiber length on printability and fiber orientation in resulting printed structures. FIG. 15 is a table of results of the evaluation. Gelatin fibers were produced by immersion rotary jet spinning (iRJS) of a polymer solution of 20% porcine gelating type 300A dissolved in DI water at 37° C. into an ethanol/water precipitation bath. The resulting gelatin fibers were between 0.5 μm and 20 μm in diameter. The gelatin fibers were then shortened by cutting and fractionating/filtering to select fiber distributions having different maximum fiber lengths and different average diameter lengths.

HA fibers were produced by iRJS of 4% HA solution into an ethanol/water precipitation bath. The resulting HA fibers were between 2 μm and 20 μm in diameter.

PCL fibers were produce by pull-spinning or RJS of a 6% PCL solution. The resulting PCL fibers were between 0.2 to 3 microns in diameter.

Carriers based on alginate and carriers based on gelatin were both employed. Information regarding a carrier based on alginate is described above with respect to Example 1. For gelatin carrier gels, porcine gelatin of 300 bloom, Type A obtained from Sigma (Sigma G2500, CAS 9005-70-8, porcine Type 300A) was used. To produce carrier gels, gelatin was dissolved in DI water or phosphate buffered saline in concentrations or 5%, 10%, or 20% (w/w) at temperatures exceeding 40° C. These gels were prepared at 40° C., 50° C., and by autoclaving gelatin powders in solution (˜115° C.). These solutions form gels upon cooling: 5% gelatin gels at ˜5-10° C., 10% gelatin gels at ˜5-20° C., and 20% gelatin gels when T<˜30° C. These carriers can therefore be printed using a temperature controlled nozzle (T>T_gel) and temperature controlled print substrate (T<T_gel). When embedded fibers have higher sol-gel temperature than the carrier, the carrier can be removed by heating (e.g., in a warm bath containing crosslinking fiber crosslinking agents).

Another carrier material that could have been employed is Pluronics F127 (poloxamer 407), a commonly used thermo-responsive sacrificial ink, with concentration-dependent sol-gel transition between ˜10° C. and 40° C. In the printed freestanding tubes and printed model ventricles described with respect to Example 3, gelled the Pluronics F127 (poloxamer 407) was used to support printed structures prior to ionic gelation of an alginate carrier. The Pluoronics F127 is removed by lowering the temperature to liquefy the Pluoronics F127.

Example carrier-fiber matches that were employed in this example included the following:

(i) PCL fiber and alginate carrier: The carrier gelation is reversible. The PCL fiber fusion was induced by temperature (T>60° C.).
(ii) Bioprotein fiber (e.g., gelatin or HA) and thermo-responsive carrier (e.g., gelatin): In this case, carrier gelation is temperature reversible and embedded fibers are crosslinked chemically or enzymatically. For example, ethanol-dried, UV-hardened gelatin fibers produced by immersion rotary jet spinning resist temperatures up to ˜50° C. They can therefore be embedded within gelatin carrier solutions, where gelatin concentration in the carrier are between 5% and 15% w/w, and have gel-sol transitions ˜10° C. to 30° C. This bioink was extruded onto a substrate that was cooled below the carrier sol-gel temperature, and the bioink gels upon cooling. The embedded fibers were then crosslinked by adding solution containing chemical or enzymatic agents. After fiber crosslinking, the print object was warmed to remove the gelatin carrier. If a pluronics F127 carrier were used instead of gelatin, the same principles apply, except the pluronics F127 becomes liquid at low temperatures and gel at higher temperatures. Therefore, a bioink including a gelatin fiber and pluronics F127 carrier would be extruded onto heated substrates (T˜10 ° C. to 40° C., depending on pluronics concentration, for example 40% pluronics F127 w/w in diH₂O has a ˜0° C. sol-gel temperature). The fiber and pluronics carrier print object would be refrigerated (4° C.) overnight to remove the pluronics carrier.

Chemical crosslinking was employed for the gelatin fibers and the hyaluronic acid fibers in different samples. Gelatin fibers and hyaluronic acid fibers were chemically crosslinked using EDC/NHS. To crosslink the gelatin fibers or hyaluronic acid fibers, extruded structures including the embedded fibers were placed into a solution of into 100% ethanol containing 10 mM EDC and 4 mM NHS, and stored them at 4° C. for 24 hrs. Gelatin and hyaluronic acid fibers were also crosslinked in a solution of 80% ethanol containing 20% DI water, also using 10 mM EDC and 4 mM NHS. Crosslinking using these solutions was conducted successfully at 4° C. and at room temperature ˜27° C.) successfully.

Gelatin fibers could be enzymatically crosslinked using calcium-independent microbial transglutaminase from a commercial source (mTG; ActivaT1; Modernist pantry, Eliot Me.) without further purification. This enzyme is supplied as a proprietary formulation with a maltodextrin support (Ajinomoto ActivaTI, 1% enzyme and 99% maltodextrin) and is reported by the manufacturer to have a specific activity of 100 U/gram. The gelatin could be crosslinked by placing the extruded structure in a solution with 4% mTG (0.04% enzyme, or 4 U/gram). The crosslinking would take ˜1 hour.

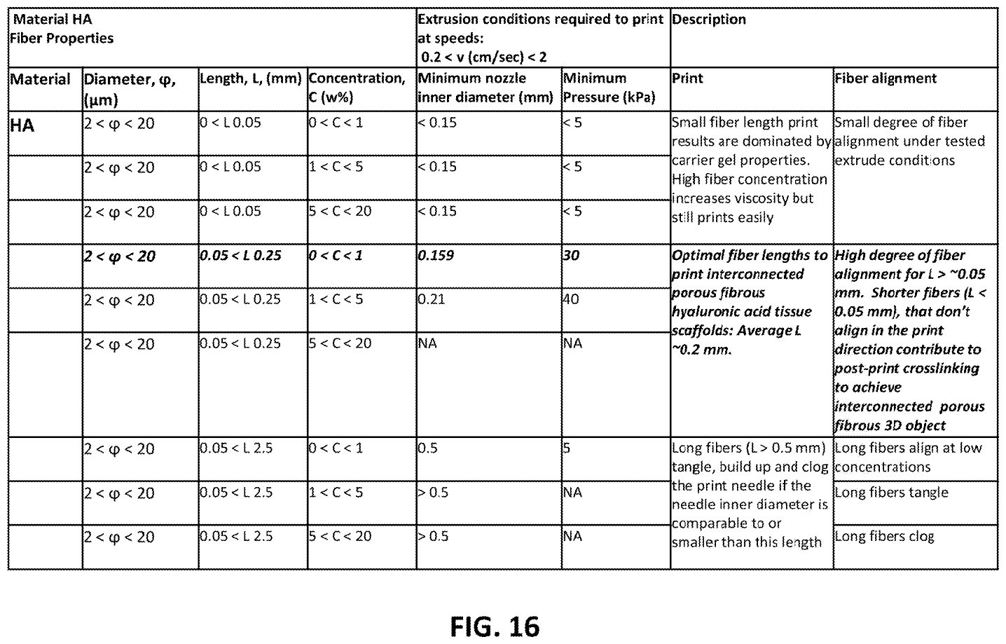

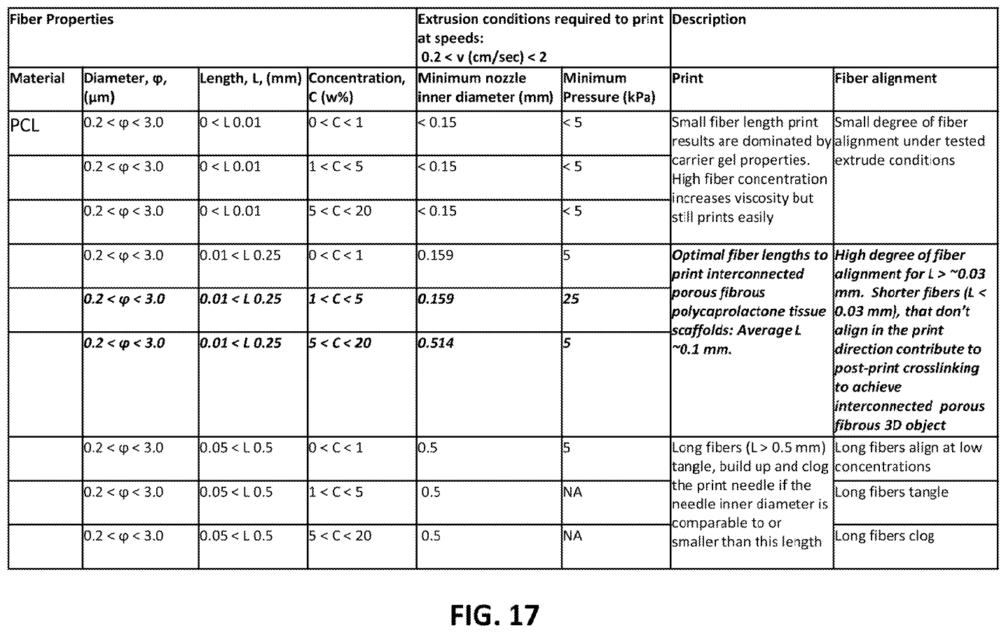

Fibers (gelatin, HA, PCL) were mixed with carriers (gelatin solution, alginate solution) at concentrations ranging between 0.1 and 20% w/w to form the bioink materials. Fiber concentrations in the bioink were given by the following: Concentration (w%)=100×(weight of fibers/weight of carrier). Optimal concentrations were defined by two criteria: (i) printability, and (ii) network formation in the printed object, and the results for each type of fiber and combination of parameters appear in FIGS. 15-17. It was determined that for print nozzle inner diameters between 0.15 mm and 0.514 mm, optimal fiber concentrations were ˜1 to 5% w/w for gelatin (fiber length ˜0.05 to 0.15 mm), 0.05 to 1.5% w/w for HA (fiber length ˜0.05 to 0.15 mm), and 5% to 25% w/w for PCL (fiber length ˜0.05 to 0.15 mm). For these “optimal fiber conditions” the bioink material solution was extruded continuously from 0.514 mm or 0.21 mm nozzles, fibers aligned in the print direction, and fiber-fiber contacts in the extruded material were sufficiently high in number to form a structurally stable network following crosslinking. The PCL fiber diameters were smaller than those of the gelatin or HA fibers (PCL˜0.2 to 3 microns vs. gelatin 0.5 to 20 microns, and HA 2 to 20 microns).

Examples of some specific recipes employed include the following:
150 mg UV-hardened gelatin fibers/1 mL DI H₂O mixed 1:1 with 5% Sodium alginate (MMW, Sigma 2133), extruded into 3% CaCl2 using 27G needle (0.21 mm inner diameter)
150 mg UV-hardened gelatin fibers/1 mL DI H2O mixed 1:1 with 5% gelatin (Sigma G2500, CAS 9005-70-8, porcine Type 300A), extruded onto a cooled substrate (T=5 ° C.) using 27G needle (0.21 mm inner diameter). UV-hardened gelatin fibers resist degradation in warm water baths (T˜20° C. to 120° C.) for sufficient time (t=1 minute to 1 hour, depending on temperature) to allow fiber crosslinking by chemical or enzymatic methods. The higher temperature of the post-print crosslinking bath degrades the 5% gelatin carrier. More specifically, UV-hardened gelatin fibers dispersed in a 5% gelatin carrier were printed at 5° C., forming a solid object. The printed object was immersed in diH₂O or PBS containing the crosslinking agent, and this solution was placed in a water bath at 20° C. for 1 hour. The temperature was then increased to 40° C. to remove remaining carrier gelatin.
50 mg HA/1 mL DI H2O mixed 1:1 with 5% Sodium alginate (MMW, Sigma 2133), extruded into 3% CaCl₂ using 27G needle (0.21 mm inner diameter)
250 mg PCL fibers/1 mL DI H2O mixed 1:1 with 5% Sodium alginate (MMW, Sigma 2133), extruded into 3% CaCl₂using 27G needle (0.21 mm inner diameter)
250 mg PCL fibers/1 mL DI H₂O mixed 1:1 with 5% gelatin (Sigma G2500, CAS 9005-70-8, porcine Type 300A), extruded onto a cooled substrate (T=5 ° C.) using 27G needle (0.21 mm inner diameter).

Bioink material having various values for the average fiber length and for the maximum fiber length were evaluated. It was determined that gelatin fibers and HA fibers longer than 0.05 mm and PCL fibers longer than 0.03 mm showed a high degree of anisotropic alignment with the extrusion direction in the resulting structures. It was also determined that an average fiber length of about 0.2 mm for the gelatin and HA fibers and an average length of about 0.1 mm for PCL fibers was optimal for creating an interconnected porous fiber scaffold after crosslinking. It was also determined that for fibers longer than 0.5 mm, the fibers tended to build up and clog the print needle/nozzle if the nozzle inner diameter was comparable or smaller than the length of the longest fibers.

### Materials:

Sodium alginate (aka Alginic acid): Low-viscosity gelatin (Sigma A1112, MW=216.12 g/mol) or high-viscosity alginate (Modernist pantry, sodium alginate, Id:1007-50) were obtained from commercial sources.

Gelatin: For gelatin carrier gels and gelatin fibers, porcine gelatin of 300 bloom, Type A obtained from Sigma (Sigma G2500, CAS 9005-70-8, porcine Type 300A). To produce carrier gels, gelatin was dissolved in DI water or phosphate buffered saline in concentrations of 5%, 10%, or 20% (w/w) at temperatures exceeding 40° C. Gels were made at 40° C., 50° C., and by autoclaving the mixed gelatin powders (˜115° C.). To produce gelatin fibers, 20% gelatin solutions were spun from an immersion rotary jet spinning system at 40° C. by into ethanol/water precipitation baths.

Hyaluronic acid: Hyaluronic acid sodium salt was obtained (from Streptococcus equi, ˜1500-1800 kDa MW, Sigma) as a powder, and dissolved in diH₂O and NaCl at various concentrations (1-4% w/w).

Polycaprolactone (PCL): PCL (Sigma-Aldrich 440744) fibers were formed by pull-spinning 6% w/w PCL dissolved in the solvent, 1,1,1,3,3,3-hexafluoro-2-propanol (HFIP; Oakwood Chemical 003409).

Microbial transglutaminase: Calcium-independent microbial transglutaminase was obtained from a commercial source (mTG; ActivaT1; Modernist pantry, Eliot Me.) and used without further purification: This enzyme is supplied as a proprietary formulation with a maltodextrin support (Ajinomoto ActivaTI, 1% enzyme and 99% maltodextrin) and is reported by the manufacturer to have a specific activity of 100 U/gram.

### EQUIVALENTS

In describing embodiments of the invention, specific terminology is used for the sake of clarity. For purposes of description, each specific term is intended to at least include all technical and functional equivalents that operate in a similar manner to accomplish a similar purpose. Additionally, in some instances where a particular embodiment of the invention includes a plurality of system elements or method steps, those elements or steps may be replaced with a single element or step; likewise, a single element or step may be replaced with a plurality of elements or steps that serve the same purpose. Further, where parameters for various properties are specified herein for embodiments of the invention, those parameters can be adjusted up or down by 1/20th, 1/10th, ⅕th, ⅓rd, ½, etc., or by rounded-off approximations thereof, unless otherwise specified. Moreover, while this invention has been shown and described with references to particular embodiments thereof, those skilled in the art will understand that various substitutions and alterations in form and details may be made therein without departing from the scope of the invention; further still, other aspects, functions and advantages are also within the scope of the invention. The contents of all references, including patents and patent applications, cited throughout this application are hereby incorporated by reference in their entirety. The appropriate components and methods of those references may be selected for the invention and embodiments thereof. Those skilled in the art will recognize, or be able to ascertain using no more than routine experimentation, many equivalents to the specific embodiments and methods described herein. Such equivalents are intended to be encompassed by the scope of the following claims.

1. A method of forming a three-dimensional tissue scaffold, the method comprising:
extruding a bioink material through a nozzle onto a support while moving the nozzle relative to the support or moving the support relative to the nozzle to form a three-dimensional structure of the bioink material, the bioink material comprising:
a plurality of polymeric fibers, each polymeric fiber having a diameter on a range of 0.1 μm to 20 μm, and each polymeric fiber comprising one or more biocompatible polymers; and
a carrier; and
cross-linking or heat fusing at least some of plurality of polymeric fibers in the three-dimensional structure of the bioink material.

2. The method of claim 1, wherein each of the plurality of polymeric fibers has a length of less than 3 mm.

3.-6. (canceled)

7. The method of claim 1, wherein the nozzle has an inner diameter in a range of 0.01 mm to 0.6 mm.

8. The method of claim 1, wherein a length of each of the plurality of polymeric fibers is less than 0.25 mm, an average length of a polymeric fiber in the plurality of polymeric fibers is between 0.05 mm and 0.25 mm, and the plurality of polymeric fibers includes polymeric fibers having lengths less than 0.05 mm.

9.-13. (canceled)

14. The method of claim 1, wherein an inner diameter of the nozzle is in a range of 0.4 mm to 2.5 mm, wherein a maximum length of a polymeric fiber in the plurality of polymeric fibers is greater than 0.25 mm and smaller than the inner diameter of the nozzle, wherein the plurality of polymeric fibers includes polymeric fibers having lengths less than 0.05 mm, and wherein a ratio of a weight of the polymeric fibers to a weight of the carrier in the bioink material is less than one.

15. The method of claim 1, wherein each of the plurality of polymeric fibers has a length less than 0.05 mm.

16. The method of claim 1, wherein at least some of the plurality of the polymeric fibers anisotropically align along a direction of extrusion in the three-dimensional structure of the fiber material.

17. The method of claim 1, wherein two or more of an inner diameter of the nozzle, an average length of polymeric fiber in the plurality of polymeric fibers, a maximum length of a polymeric fiber in the plurality of polymeric fibers, a ratio of weight of polymeric fibers to weight of carrier in the bioink material and a composition of the plurality of polymeric fibers are selected such that at least some of the plurality of polymeric fibers anisotropically align along a direction of extrusion in the three-dimensional structure of the fiber material.

18. The method of claim 1 wherein the bioink material is extruded at ambient temperature.

19. The method of claim 1, further comprising providing the bioink material.

20. The method of claim 19, wherein providing the bioink material includes disposing the plurality of polymeric fibers in the carrier less than about 30 minutes prior to a beginning of extruding the bioink material onto the support.

21. The method of claim 20, wherein providing the bioink material includes:
mechanically breaking down a polymeric fibrous material to reduce average polymeric fiber length;
fractionating a slurry including the polymeric fibrous material to obtain a desired distribution of polymeric fiber lengths;
drying the fractionated slurry leaving the plurality of polymeric fibers having the desired distribution of polymeric fiber lengths; and
suspending the plurality of polymeric fibers having the desired distribution of polymeric fiber lengths in the carrier.

22. The method of claim 1, wherein the cross-linking includes chemical cross-linking or enzymatic cross-linking

23.-28. (canceled)

29. The method of claim 1, wherein the carrier comprises a hydrogel forming solution.

30.-41. (canceled)

42. The method of claim 1, wherein movement of the nozzle relative to the support or movement of the support relative to the nozzle to form a three-dimensional structure of the bioink material includes using a 3-D printing system or additive manufacturing system to control relative movement of the nozzle and the support.

43. A method of forming a three-dimensional engineered tissue comprising:
providing a three-dimensional tissue scaffold produced using the method of claim 1;
seeding the scaffold with cells; and
culturing the cells under suitable conditions to form a tissue, thereby forming a three-dimensional engineered tissue.

44. A method of forming an engineered food product comprising:
providing a three-dimensional tissue scaffold produced using the method of claim 1;
seeding the scaffold with muscle cells; and
culturing the cells under suitable conditions to form a muscle tissue, thereby forming a three-dimensional engineered food product.

45. A bioink material for use with a three-dimensional printer or an additive manufacturing system, the bioink comprising:
a plurality of polymeric fibers having an average length in a range of 0.07 mm to 0.25 mm, each polymeric fiber having a diameter on a range of 0.05 mm and 0 3 mm, and each polymeric fiber comprising one or more biocompatible polymers; and
a carrier comprising a hydrogel forming solution, wherein the average length of the plurality of polymeric fibers results in at least some of the polymeric fibers being preferentially oriented along an extrusion direction when the bioink is extruded from a three-dimensional printer or additive manufacturing system.

46.-52. (canceled)

53. A kit for forming the bioink material of claim 45, the kit comprising:
the plurality of polymeric fibers; and
a carrier forming material such that mixing the carrier forming material with water forms the carrier.
